SEQUENCE LISTING (32 sequences)

SEQ ID NO: 1 - protein, 13 aa, Artificial Sequence
  Synthetic Sequence
AASGFDFSSYGVP

SEQ ID NO: 2 - protein, 17 aa, Artificial Sequence
  synthetic sequence
YIDPIFGPTYYADEVKG

SEQ ID NO: 3 - protein, 14 aa, Artificial Sequence
  synthetic construct
ARDYSYGYAYALDI

SEQ ID NO: 4 - protein, 11 aa, Artificial Sequence
  synthetic construct
QASQRISPYLA

SEQ ID NO: 5 - protein, 8 aa, Artificial Sequence
  synthetic construct
SRASKLAS

SEQ ID NO: 6 - protein, 12 aa, Artificial Sequence
  synthetic construct
QSYYVHTSSGYA

SEQ ID NO: 7 - protein, 13 aa, Artificial Sequence
  synthetic construct
KASGGTFSSYAIS

SEQ ID NO: 8 - protein, 17 aa, Artificial Sequence
  synthetic construct
LIIPSFDTAGYAQKFQG

SEQ ID NO: 9 - protein, 13 aa, Artificial Sequence
  synthetic construct
ARAEHSSTGTFDY

SEQ ID NO: 10 - protein, 11 aa, Artificial Sequence
  synthetic construct
RASQGISSWLA

SEQ ID NO: 11 - protein, 8 aa, Artificial Sequence
  synthetic construct
SAASSLQS

SEQ ID NO: 12 - protein, 9 aa, Artificial Sequence
  synthetic construct
QQANHLPFT

SEQ ID NO: 13 - protein, 121 aa, Artificial Sequence
  synthetic construct
EVQLVESGGGLVQPGGSLRLSCAASGFDFSSYGVPWVRKAPGKGLEWVGYIDPIFGPTYYADEVKGRFTISADDSKNSLYLQMNSLKTEDTAVYYCARDYSYGYAYALDIWGQGTLVTVSS

SEQ ID NO: 14 - protein, 110 aa, Artificial Sequence
  synthetic construct
RIVMTQTPLSLSVTPGQPASISCQASQRISPYLAWYLDKPGQPPQLLISRASKLASGVPDRFSGSGSGTDFTLKISRVEAEDVGVYYCQSYYVHTSSGYAFGGGTKVEIK

SEQ ID NO: 15 - protein, 330 aa, Artificial Sequence
  synthetic construct
ASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 16 - protein, 107 aa, Artificial Sequence
  synthetic construct
RTVAAPSVFIFPPSDKQLKSGTARVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 120 aa, Artificial Sequence
  synthetic construct
QVQLVQSGAEVKKPGSSVKVSCKASGGTFSSYAISWVRYAPGQGLEWMGLIIPSFDTAGYAQKFQGRVAITVDESTSTAYMELSSLRSEDTAVYYCARAEHSSTGTFDYWGRGTLVTVSS

SEQ ID NO: 18 - protein, 107 aa, Artificial Sequence
  synthetic construct
DIQMTQSPSSVSASVGDRVTITCRASQGISSWLAWYQRKPGDAPKLLISAASSLQSGVPSRFSGSGSGTDFTLTISSLQPEDFATYYCQQANHLPFTFGGGTKVEIK

SEQ ID NO: 19 - protein, 330 aa, Artificial Sequence
  synthetic construct
ASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVMHEALHNHYTQKSLSLSPGK

SEQ ID NO: 20 - protein, 105 aa, Artificial Sequence
  synthetic construct
GQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 21 - protein, 451 aa, Artificial Sequence
  synthetic construct
EVQLVESGGGLVQPGGSLRLSCAASGFDFSSYGVPWVRKAPGKGLEWVGYIDPIFGPTYYADEVKGRFTISADDSKNSLYLQMNSLKTEDTAVYYCARDYSYGYAYALDIWGQGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 22 - protein, 217 aa, Artificial Sequence
  synthetic construct
RIVMTQTPLSLSVTPGQPASISCQASQRISPYLAWYLDKPGQPPQLLISRASKLASGVPDRFSGSGSGTDFTLKISRVEAEDVGVYYCQSYYVHTSSGYAFGGGTKVEIKRTVAAPSVFIFPPSDKQLKSGTARVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 450 aa, Artificial Sequence
  synthetic construct
QVQLVQSGAEVKKPGSSVKVSCKASGGTFSSYAISWVRYAPGQGLEWMGLIIPSFDTAGYAQKFQGRVAITVDESTSTAYMELSSLRSEDTAVYYCARAEHSSTGTFDYWGRGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVMHEALHNHYTQKSLSLSPGK

SEQ ID NO: 24 - protein, 212 aa, Artificial Sequence
  synthetic construct
DIQMTQSPSSVSASVGDRVTITCRASQGISSWLAWYQRKPGDAPKLLISAASSLQSGVPSRFSGSGSGTDFTLTISSLQPEDFATYYCQQANHLPFTFGGGTKVEIKGQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 25 - protein, 1413 aa, Artificial Sequence
  synthetic construct
ATGGAGACGGACACTCTGCTCCTGTGGGTGCTCCTGCTTTGGGTACCGGGTTCAACGGGAGAGGTGCAGCTGGTGGAGTCTGGGGGAGGCTTGGTCCAGCCTGGAGGGTCCCTGAGACTCTCCTGTGCTGCTTCTGGATTCGACTTCAGTAGTTATGGAGTGCCCTGGGTCCGCAAGGCTCCAGGGAAGGGGCTGGAGTGGGTTGGCTACATTGATCCTATTTTTGGTCCCACATACTACGCAGACGAGGTGAAGGGCAGATTCACCATCTCAGCTGATGATTCAAAGAACTCACTGTATCTGCAAATGAACAGCCTGAAAACCGAGGACACGGCCGTGTATTACTGTGCGAGAGACTATAGTTATGGTTATGCTTATGCTCTCGACATCTGGGGCCAGGGAACCCTGGTCACCGTCTCCTCAGCTAGCACCAAGGGCCCATCGGTCTTCCCCCTGGCACCCTCCTCCAAGAGCACCTCTGGGGGCACAGCGGCCCTGGGCTGCCTGGTCGCCGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACGAGAGAGTTGAGCCCAAATCTTGTGACAAAACTCACACATGCCCACCGTGCCCAGCACCTGAAGCCGCAGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTATGTGGACGGCGTGGAGGTGCATAATGCCAAGACAAAGCCGCGGGAGGAGCAGTACAACAGCACGTACCGTGTGGTCAGCGTCCTCACCGTCCTGCACCAAGACTGGCTGAATGGCAAGGAGTACAAGTGCAAGGTCTCCAACAAAGCCCTCGCCGCCCCCATCGAGAAAACCATCTCCAAAGCCAAAGGGCAGCCCCGAGAACCACAGGTGTACACCCTGCCCCCATCCCGGGGGGACATGACCAAGAACCAAGTCCAGCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCGCTTCCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGCAAA

SEQ ID NO: 26 - protein, 711 aa, Artificial Sequence
  synthetic construct
ATGGAAACTGACACCCTGCTGCTCTGGGTACTGCTCCTTTGGGTTCCTGGGAGCACAGGCCGGATTGTGATGACCCAGACTCCACTCTCTCTGTCCGTCACCCCTGGACAGCCGGCCTCCATCTCCTGCCAGGCCAGTCAGAGAATTAGTCCCTACTTAGCCTGGTACCTGGACAAGCCAGGCCAGCCTCCACAGCTCCTGATCTCCCGGGCATCCAAACTGGCATCTGGAGTGCCAGATAGGTTCAGTGGCAGCGGGTCAGGGACAGATTTCACACTGAAAATCAGCCGGGTGGAGGCTGAGGATGTTGGGGTTTATTACTGCCAAAGTTATTATGTTCACACTAGTAGTGGTTATGCTTTCGGCGGAGGGACCAAGGTGGAGATCAAACGGACCGTGGCTGCACCATCTGTCTTCATCTTCCCGCCATCTGATAAGCAGTTGAAATCTGGAACTGCCAGAGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACTCATCAGGGCCTGAGCTCGCCCGTCACAAAGAGCTTCAACAGGGGAGAGTGC

SEQ ID NO: 27 - protein, 1410 aa, Artificial Sequence
  synthetic construct
ATGGAAACCGATACGCTCCTGCTGTGGGTTCTCCTCTTGTGGGTCCCCGGCTCTACCGGGCAGGTCCAGCTCGTGCAGAGTGGCGCCGAGGTCAAAAAACCCGGTTCAAGCGTGAAGGTGTCTTGTAAAGCATCTGGAGGAACCTTTAGTTCCTACGCCATTAGTTGGGTGAGGTACGCTCCCGGCCAGGGCTTGGAATGGATGGGTTTGATTATTCCCAGCTTTGATACAGCTGGATACGCGCAGAAGTTCCAGGGACGCGTGGCCATCACCGTGGATGAAAGCACTTCAACTGCCTACATGGAACTGTCATCCTTGAGAAGCGAGGATACTGCTGTTTACTACTGCGCTAGGGCAGAGCACTCCTCCACCGGGACCTTCGACTATTGGGGTCGAGGTACTCTCGTGACCGTGAGCAGCGCTAGCACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGGCCACCGGCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAGAGTTGAGCCCAAATCTTGTGACAAAACTCACACATGCCCACCGTGCCCAGCACCTGAAGCCGCAGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTATGTGGACGGCGTGGAGGTGCATAATGCCAAGACAAAGCCGCGGGAGGAGCAGTACAACAGCACGTACCGTGTGGTCAGCGTCCTCACCGTCCTGCACCAAGACTGGCTGAATGGCAAGGAGTACAAGTGCAAGGTCTCCAACAAAGCCCTCGCCGCCCCCATCGAGAAAACCATCTCCAAAGCCAAAGGGCAGCCCCGAGAACCACAGGTGTCCACCCTGCCCCCATCCCGGGAGGAGATGACCAAGAACCAAGTCAGCCTGATGTGCCTGGTCTATGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATTCCGTGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGCAAA

SEQ ID NO: 28 - protein, 696 aa, Artificial Sequence
  synthetic construct
ATGGAGACAGACACACTCCTGCTATGGGTACTGCTGCTCTGGGTTCCAGGATCCACTGGTGACATCCAGATGACACAGTCACCTTCAAGCGTCTCCGCCTCCGTGGGAGACAGGGTTACTATTACATGTAGGGCCAGCCAGGGGATCTCTTCATGGCTGGCGTGGTACCAACGGAAGCCAGGCGACGCCCCCAAGCTCCTTATCTCCGCTGCCTCCTCTCTGCAGTCCGGAGTTCCCTCCCGCTTCAGCGGTAGCGGGTCAGGCACTGACTTCACCCTTACAATCTCTTCTCTGCAACCTGAGGACTTCGCCACATATTATTGCCAGCAGGCAAACCATTTGCCATTTACTTTTGGCGGAGGTACTAAGGTTGAGATTAAAGGCCAGCCTAAAGCTGCCCCTAGCGTTACCCTTTTCCCACCGAGCTCCGAGGAGCTGCAGGCCAATAAAGCAACCTTGGTCTGCTACATATCAGATTTTTACCCTGGCGCCGTGACCGTAGCATGGAAAGCTGATTCATCCCCTGTGAAGGCCGGTGTTGAAACTACAACCCCTTCCAAACAATCTAACAATAAATACGCGGCATGGTCCTACCTGTCCTTGACACCCGAGCAGTGGAAATCTCACAGATCTTACAGCTGCCAGGTCACCCACGAGGGGAGCACTGTGGAGAAGACCGTCGCGCCCACTGAGTGC

SEQ ID NO: 29 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 30 - protein, 149 aa, Artificial Sequence
  synthetic construct
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQHHHHHH

SEQ ID NO: 31 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 32 - protein, 142 aa, Artificial Sequence
  synthetic construct
HHHHHHGGGGSMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPGGGGSHHHHHH

(19) United States
(12) Patent Application Publication
(10) Pub. No.: US 2022/0227860 A1
(43) Pub. Date: Jul. 21, 2022

(54) TIGIT AND PD-1/TIGIT-BINDING MOLECULES

(71) Applicant: Eli Lilly and Company, Indianapolis, IN (US)

(72) Inventors: Yiqing FENG, Carmel, IN (US); Naresh KUMAR, Warrington, PA (US); James David PANCOOK, San Diego, CA (US); Stephanie Marie TRUHLAR, Carlsbad, CA (US); Yang ZHAO, Lexington, PA (US)

(21) Appl. No.: 17/613,675

(22) PCT Filed: May 22, 2020
(86) PCT No.: PCT/US2020/034158

§ 371 Date: Nov. 23, 2021

Related U.S. Application Data
(60) Provisional application No. 62/853,816, filed on May 29, 2019.

(51) Int. Cl. C07K 16/28; A61P 35/00

(52) U.S. Cl. CPC C07K 16/28; A61K 2039/507; A61P 35/00

(57) ABSTRACT

The present invention relates to polypeptide molecules that bind to human TIGIT, and to polypeptide molecules that bind to human PD-1 and human TIGIT, and are useful for treating solid tumors, alone and in combination with chemotherapy and/or ionizing radiation.

The present invention is in the field of medicine. Particularly, the present invention relates to novel polypeptide molecules that antagonize human TIGIT or that antagonize both human TIGIT and human PD-1, compositions comprising such polypeptide molecules, and methods of using such polypeptide molecules for the treatment of solid tumors, alone or in combination with chemotherapy and other cancer therapeutics.

Immune checkpoints are a group of membrane proteins expressed on immune cells (e.g., T cells & dendritic cells), including multiple co-inhibitory and co-stimulatory receptors, that play an important role in the regulation of the adaptive immune response. Checkpoints include human programmed cell death ligand (PD-1) (NCBI NP_005009.2) and human T cell immunoreceptor with Ig and ITIM domains (TIGIT) (NCBI NP_776160.2).

The interaction between PD-1 and its ligands, programmed cell death ligand 1 (PD-L1) and programmed cell death ligand 2 (PD-L2), provides an inhibitory signal that has been shown to play a key role in tumor immune escape and the immunosuppression that occurs in the tumor microenvironment. While the blockade of PD-1 inhibitory signaling with anti-PD-1 antibodies and/or anti-PD-L1 antibodies is clinically validated and has led to significant clinical advances for the treatment of certain cancers, there are many patients who either do not respond, relapse, acquire resistance to PD-1 or PD-L1 antibody treatment(s), or otherwise are intolerant to treatment.

TIGIT is a coinhibitory receptor expressed, like PD-1, on activated and exhausted T cells. TIGIT binds to the Poliovirus receptor (PVR, also known as CD155) on tumor cells, and enables reverse signaling into tumor cells that results in the secretion of T-cell-suppressive cytokines. Although CD155 is considered the dominant ligand for TIGIT, TIGIT can also interact with CD112 and CD113 (Blake et al., Clin Cancer Res; 2016; 22(21): 5182-5188). The role of TIGIT as an inhibitory immune checkpoint receptor has been studied. TIGIT is part of the CD226/TIGIT pathway, in which TIGIT not only competes with CD226 a co-stimulatory immune receptor for binding to CD155 but also directly interacts with CD226 in the cell membrane, and blocks CD226 homodimerization. (Blake et al., S, Clin Cancer Res; 2016; 22(21): 5182-5188; Johnston et al., Cancer Cell 2014; 26: 923-937; Mahnke et al, Journal of Investigative Dermatology 2016; 136: 9-11).

Anti-TIGIT antibodies are known in the art, including those which are disclosed in US 2016/0355589, US 2017/143825, US 2017/088613, US 2016/376365, US 2018/169238, US 2016/176963, and US 2019/100591. However, no anti-human TIGIT antibody has received regulatory approval for therapeutic use in humans, alone or in combination with an anti-human PD-L1 or an anti-human PD-1 antibody. Furthermore, no bispecific antibody targeting TIGIT and PD-1 or TIGIT and PD-L1 has received regulatory approval for therapeutic use in humans. Thus, there exists a need for additional treatments that target immune checkpoint pathways.

Accordingly, the present invention is directed to novel anti-human TIGIT antibodies and novel anti-human TIGIT/anti-human PD-1 bispecific antibodies. Furthermore, unlike other anti-human TIGIT antibodies, the antibodies of the present invention are effector function null, i.e., are engineered to minimize Fc receptor binding. Thus, unlike other anti-human TIGIT antibodies, the antibodies of the present invention do not contain a native human IgG1 framework that can contribute to T regulatory cell depletion and immune response adverse events. In addition, the anti-human TIGIT/anti-human PD-1 bispecific antibodies of the present invention contain different types of light chains, wherein the anti-human TIGIT arm light chain is a kappa light chain, and the anti-human PD-1 light chain is a lambda light chain, which facilitates heteromab bispecific antibody formation by decreasing the potential for light chain-light chain dimerization.

The preparation of bispecific molecules is generally known to be an unpredictable endeavor. For example, coexpressing two heavy chains and two light chains to generate an IgG bispecific antibody can result in some missassembly and unwanted byproducts, ameheterodimeric interactions within antibody Fabs (Lewis S M et al., Nature Biotechnology 2014; 32: 191-202; Leaver-Fay A, et al., Structure 2016; 24: 641-651). Thus, the present invention provides an anti-human TIGIT/anti-human PD-1 bispecific molecule that minimized Fc receptor binding, minimizes oxidation, facilitates heteromab assembly, and is cross-reactive with human TIGIT/PD-1 and cynomolgous TIGIT/PD-1, and exhibits in vivo efficacy in an established tumor model.

Surprisingly, an anti-human TIGIT/anti-human PD-1 bispecific antibody of the invention demonstrates significant in vivo anti-tumor efficacy, when compared to anti-human PD-1 and anti-human TIGIT antibody combination therapy. More surprisingly, treatment with a bispecific antibody of the invention results in an increase in the percentage of both CD226+ CD8 T cells and CD226+ NK cells, which may contribute to the significant in vivo efficacy observed.

The present invention also provides a polypeptide molecule that binds to human TIGIT (SEQ ID NO:31) or to a human TIGIT extracellular domain, e.g., SEQ ID NO: 32, comprising the heavy and light complementarity determining region (CDR) amino acid sequences of SEQ ID NOS:1-6 (see Table 1). In one embodiment, the polypeptide further comprises the CDR amino acid sequences of SEQ ID NOS: 7-12, wherein the polypeptide molecule also binds to human PD-1 (SEQ ID NO: 29), or to a PD-1 extracellular domain, e.g., SEQ ID NO: 30.

In one embodiment, the polypeptide molecule is a scFv molecule. In another embodiment, the polypeptide molecule is a polyspecific scFv molecule. In another embodiment, the polyspecific scFv molecule is a bispecific scFv molecule.

In one embodiment, the polypeptide molecule is an antibody, or a human TIGIT-binding fragment thereof, comprising three HCDRs having the amino acid sequences of SEQ ID NOS: 1-3, respectively, and three LCDRs having the amino acid sequences of SEQ ID NOS: 4-6, respectively. In another embodiment, the polypeptide molecule is an antibody. In another embodiment, the antibody is a mono-specific antibody. In another embodiment, the antibody is a polyspecific antibody. In another embodiment, the antibody is a bispecific antibody that also binds to human PD-1.

In another embodiment, the polypeptide molecule is an antibody or human TIGIT-binding fragment thereof comprising a heavy chain variable region having the amino acid sequence of SEQ ID NO: 13 and a light chain variable region having the amino acid sequence of SEQ ID NO: 14. In another embodiment, the polypeptide molecule is an antibody comprising a heavy chain having the amino acid sequence of SEQ ID NO: 21 and a light chain having the amino acid sequence of SEQ ID NO: 22.

In another embodiment, the antibody or human TIGIT-binding fragment thereof also binds to human PD-1 (SEQ ID NO: 31), or to a human TIGIT extracellular domain, e.g., SEQ ID NO: 32, and to human PD-1 (SEQ ID NO: 29), or to a human PD-1 extracellular domain, e.g., SEQ ID NO: 30, and further comprises three HCDRs having the amino acid sequence of SEQ ID NOS: 7-9, respectively, and three LCDRs having the amino acid sequences of SEQ ID NOS: 10-12, respectively.

In another embodiment, the polypeptide molecule is an antibody, or a human TIGIT and human PD-1 binding fragment thereof, comprising: a first heavy chain variable region having the amino acid sequence of SEQ ID NO: 13; a first light chain variable region having the amino acid sequence of SEQ ID NO: 14; a second heavy chain variable region having the amino acid sequence of SEQ ID NO: 17; and a second light chain variable region having the amino acid sequence of SEQ ID NO: 18.

In another embodiment, the polypeptide molecule is an antibody comprising: a first heavy chain having the amino acid sequence of SEQ ID NO:21; a first light chain having the amino acid sequence of SEQ ID NO:22; a second heavy chain having the amino acid sequence of SEQ ID NO:23; and a second light chain having the amino acid sequence of SEQ ID NO:24.

The present invention also provides a mammalian cell capable of expressing the polypeptide molecule of the invention.

The present invention also provides a DNA molecule comprising a polynucleotide encoding one or more of the amino acid sequences of SEQ ID NO:21, SEQ ID NO: 22, SEQ ID NO: 23 and SEQ ID NO: 24. The present invention also provides a DNA molecule of claim 17, wherein the polynucleotide comprises one or more of the DNA sequences of SEQ ID NO:25, SEQ ID NO: 26, SEQ ID NO: 27 and SEQ ID NO: 28.

The present invention also provides a mammalian cell comprising a DNA molecule of the invention. The present invention also provides a process for producing an antibody, comprising cultivating a mammalian cell of the invention, and recovering the polypeptide molecule. The present invention also provides the polypeptide molecule produced by the method.

The present invention also provides a pharmaceutical composition comprising a polypeptide molecule of the invention, and an acceptable carrier, diluent, or excipient.

The present invention also provides a method of treating a solid tumor cancer comprising administering to a human patient in need thereof, an effective amount of a polypeptide molecule of the invention. In one embodiment, the solid tumor cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. In another embodiment, the lung cancer is non-small cell lung cancer or small cell lung cancer. In another embodiment, the breast cancer is triple-negative breast cancer. In another embodiment, the polypeptide molecule is administered in simultaneous, separate, or sequential combination with ionizing radiation. In another embodiment, the polypeptide molecule is administered in simultaneous, separate, or sequential combination with one or more chemotherapeutic agents.

The present invention also provides a polypeptide molecule of the invention, for use in therapy. In one embodiment, the use is use in treating a solid tumor cancer. In another embodiment, the solid tumor cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. In another embodiment, the lung cancer is non-small cell lung cancer or small cell lung cancer. In another embodiment, the breast cancer is triple-negative breast cancer. In another embodiment, the polypeptide molecule is administered in simultaneous, separate, or sequential combination with ionizing radiation. In another embodiment, the polypeptide molecule is administered in simultaneous, separate, or sequential combination with one or more chemotherapeutic agents.

The present invention also provides for the use of a polypeptide molecule of the invention in the manufacture of a medicament for treating a solid tumor cancer. In one embodiment, the use is use in treating a solid tumor cancer. In another embodiment, the solid tumor cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. In another embodiment, the lung cancer is non-small cell lung cancer or small cell lung cancer. In another embodiment, the breast cancer is triple-negative breast cancer. In another embodiment, the polypeptide molecule is administered in simultaneous, separate, or sequential combination with ionizing radiation. In another embodiment, the polypeptide molecule is administered in simultaneous, separate, or sequential combination with one or more chemotherapeutic agents.

In one embodiment, an antibody of the present invention is a bispecific antibody. The bispecific antibodies of the present invention are designed to favor heterodimeric pairing of the two distinct heavy chains and disfavor formation of homodimers. Preferably, the bispecific antibodies described herein contain an Fc portion that is derived from human IgG1. Human IgG1 is known to bind to the proteins of the Fc-gamma receptor (FcγR) family as well as C1q. IgG1 binding to an FcγR or C1q induces antibody-dependent cellular cytotoxicity (ADCC) and complement-dependent cytotoxicity (CDC), respectively. Therefore, preferably, the antibodies described herein are a human IgG1 engineered to reduce the binding of the antibody to an FcγR as well as C1q. Preferably, amino acid substitutions of positions L234A, L235A and P329A in EU numbering are introduced into the CH2 region to reduce the binding of the antibody to an FcγR as well as C1q. Optionally, amino acid substitution of position N297Q in EU numbering is introduced to further reduce the ADCC and CDC activities of the antibody.

The framework and CDR sequences in each of the antibodies for which sequences are set forth herein are annotated using annotation rules in agreement with the method of North, et al. J. Mol. Biol. 2011; 406: 228-256.

The present invention also provides an antibody that binds to human TIGIT (SEQ ID NO:31), or to a TIGIT extracellular domain, e.g., SEQ ID NO: 32, comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) a light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6.

The present invention also provides an antibody comprising:
a) a heavy chain variable region having the amino acid sequence of SEQ ID NO:13; and
b) a light chain variable region having the amino acid sequence of SEQ ID NO:14.

The present invention also provides an antibody comprising:

a) a heavy chain having the amino acid sequence of SEQ ID NO:21; and

b) a light chain having the amino acid sequence of SEQ ID NO:22.

In one embodiment, the heavy chain of the antibody forms at least one disulfide bond with the light chain of the antibody, and the two heavy chains of the antibody form at least one disulfide bond.

In another embodiment, the antibody is a human IgG1 engineered to reduce the binding of the antibody to an Fc gamma receptor.

The present invention also provides a DNA molecule comprising a polynucleotide encoding for at least one polypeptide having the amino acid sequence of SEQ ID NO:21 and the amino acid sequence of SEQ ID NO:22. In a preferred embodiment, the DNA molecule comprises a polynucleotide comprising at least one of SEQ ID NO: 25 and SEQ ID NO: 26.

The present invention also provides a mammalian cell comprising a DNA molecule comprising a polynucleotide encoding for at least one polypeptide having the amino acid sequence of SEQ ID NO:21 and the amino acid sequence of SEQ ID NO:22.

The present invention also provides a process for producing an antibody comprising cultivating a mammalian cell capable of expressing the antibody and recovering the antibody, comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6.

In one embodiment, the heavy chain of the antibody forms at least one disulfide bond with the light chain of the antibody, and the two heavy chains of the antibody form at least one disulfide bond.

The present invention also provides a process for producing an antibody comprising cultivating a mammalian cell capable of expressing the antibody and recovering the antibody, comprising:
a) a heavy chain variable region having the amino acid sequence of SEQ ID NO:13; and
b) a light chain variable region having the amino acid sequence of SEQ ID NO:14.

The present invention also provides a process for producing an antibody comprising cultivating a mammalian cell capable of expressing the antibody and recovering the antibody, comprising:

a) a heavy chain having the amino acid sequence of SEQ ID NO:21; and

b) a light chain having the amino acid sequence of SEQ ID NO:22.

The present invention also provides a process for producing an antibody comprising cultivating a mammalian cell capable of expressing the antibody and recovering the antibody; wherein the antibody is a human IgG1 engineered to reduce the binding of the antibody to an Fc gamma receptor.

The present invention also provides an antibody produced by a process comprising cultivating a mammalian cell capable of expressing the antibody and recovering the antibody, the antibody comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) a light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6.

In one embodiment, the heavy chain of the antibody forms at least one disulfide bond with the light chain of the antibody, and the two heavy chains of the antibody form at least one disulfide bond.

The present invention also provides an antibody produced by a process comprising cultivating a mammalian cell capable of expressing the antibody and recovering the antibody, comprising:
a) a heavy chain variable region having the amino acid sequence of SEQ ID NO:13;
b) a light chain variable region having the amino acid sequence of SEQ ID NO:14.

The present invention also provides an antibody produced by a process comprising cultivating a mammalian cell capable of expressing the antibody and recovering the antibody, comprising:

a) a heavy chain having the amino acid sequence of SEQ ID NO:21;

b) a light chain having the amino acid sequence of SEQ ID NO:22.

The present invention also provides a pharmaceutical composition comprising an antibody, wherein the antibody binds to human TIGIT (SEQ ID NO:31), or to a human TIGIT extracellular domain, e.g., SEQ ID NO: 32, comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) a light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6,

and an acceptable carrier, diluent, or excipient.

In one embodiment, the heavy chain of the antibody forms at least one disulfide bond with the light chain of the antibody, and the two heavy chains of the antibody form at least one disulfide bond.

The present invention also provides a pharmaceutical composition comprising an antibody comprising:
a) a heavy chain variable region having the amino acid sequence of SEQ ID NO:13;
b) a light chain variable region having the amino acid sequence of SEQ ID NO:14,

and an acceptable carrier, diluent, or excipient.

The present invention also provides a pharmaceutical composition comprising an antibody comprising:

a) a heavy chain having the amino acid sequence of SEQ ID NO:21;

b) a light chain having the amino acid sequence of SEQ ID NO:22,

and an acceptable carrier, diluent, or excipient.

In one embodiment, antibody is a human IgG1 engineered to reduce the binding of the antibody to an Fc gamma receptor.

The present invention also provides a method of treating cancer comprising administering to a human patient in need thereof, an effective amount of an antibody, wherein the antibody binds to human TIGIT (SEQ ID NO:31), or a human TIGIT extracellular domain, e.g., SEQ ID NO: 32, comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) a light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6.

In one embodiment, the heavy chain of the antibody forms at least one disulfide bond with the light chain of the antibody, and the two heavy chains of the antibody form at least one disulfide bond.

The present invention also provides a method of treating cancer comprising administering to a human patient in need thereof, an effective amount of an antibody, comprising:
a) a heavy chain variable region having the amino acid sequence of SEQ ID NO:13; and
b) a light chain variable region having the amino acid sequence of SEQ ID NO:14.

The present invention also provides a method of treating cancer comprising administering to a human patient in need thereof, an effective amount of an antibody, comprising:

a) a heavy chain having the amino acid sequence of SEQ ID NO:21;

b) a light chain having the amino acid sequence of SEQ ID NO:22.

In one embodiment, the antibody is a human IgG1 engineered to reduce the binding of the antibody to an Fc gamma receptor.

The present invention also provides methods of treatment and methods for use.

The present invention also provides a method of treating cancer comprising administering to a human patient in need thereof, an effective amount of an antibody described herein, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma.

The present invention also provides a method of treating cancer comprising administering to a human patient in need thereof, an effective amount of an antibody described herein, wherein the cancer is non-small cell lung cancer, or small cell lung cancer. The present invention further provides a method of treating cancer comprising administering to a human patient in need thereof, an effective amount of an antibody described herein, wherein the cancer is triple negative breast cancer.

The present invention also provides a method of treating cancer comprising administering to a human patient in need thereof, an effective amount of an antibody described herein, wherein the antibody is administered in combination with ionizing radiation.

The present invention also provides a method of treating cancer comprising administering to a human patient in need thereof, an effective amount of an antibody described herein, wherein the antibody is administered in combination with one or more chemotherapeutic agents.

The present invention also provides a method of treating cancer comprising administering to a human patient in need thereof, an effective amount of an antibody described herein, wherein the antibody is administered in combination with ionizing radiation and one or more chemotherapeutic agents.

In one embodiment, the present invention also provides a method of treating cancer, comprising administering an effective amount of a bispecific antibody disclosed herein in simultaneous, separate, or sequential combination with one or more anti-tumor agents. Non-limiting examples of anti-tumor agents include ramucirumab, necitumumab, olaratumab, gemcitabine, pemetrexed, galunisertib, abemaciclib, cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), cetuximab, an EGFR inhibitor, a Raf inhibitor, a B-Raf inhibitor, a CDK4/6 inhibitor, a CDK7 inhibitor, an idoleamine 2,3-dioxygenase inhibitor, a TGFβ inhibitor, a TGFβ receptor inhibitor, IL-10, and pegylated IL-10 (e.g., pegilodecakin).

The present invention also provides an antibody for use in treating cancer, wherein the antibody binds to human TIGIT (SEQ ID NO:31), or to a human TIGIT extracellular domain, e.g., SEQ ID NO: 32, comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) a light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6.

In one embodiment, the heavy chain of the antibody forms at least one disulfide bond with the light chain of the antibody, and the two heavy chains of the antibody form at least one disulfide bond.

The present invention also provides an antibody for use in treating cancer, comprising:
a) a heavy chain variable region having the amino acid sequence of SEQ ID NO:13; and
b) a light chain variable region having the amino acid sequence of SEQ ID NO:14.

The present invention also provides an antibody for use in treating cancer, comprising:

a) a heavy chain having the amino acid sequence of SEQ ID NO:21; and

b) a light chain having the amino acid sequence of SEQ ID NO:22.

In one embodiment, the antibody is a human IgG1 engineered to reduce the binding of the antibody to an Fc gamma receptor.

The present invention also provides an antibody for use in treating cancer, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. The present invention further provides an antibody for use in treating lung cancer, wherein the lung cancer is non-small cell lung cancer or small cell lung cancer. The present invention also provides an antibody for use in treating breast cancer, wherein the breast cancer is triple-negative breast cancer.

In one embodiment, the antibody is administered in simultaneous, separate, or sequential combination with ionizing radiation. In another embodiment, the antibody is administered in simultaneous, separate, or sequential combination with one or more chemotherapeutic agents. In another embodiment, the antibody is administered in simultaneous, separate, or sequential combination with ionizing radiation and one or more chemotherapeutic agents.

The present invention also provides a pharmaceutical composition comprising an antibody for use in treating cancer, wherein the antibody binds to human TIGIT (SEQ ID NO:31), or a human TIGIT extracellular domain, e.g., SEQ ID NO: 32, comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) a light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6,

and an acceptable carrier, diluent, or excipient.

In one embodiment, the heavy chain of the antibody forms at least one disulfide bond with the light chain of the antibody, and the two heavy chains of the antibody form at least one disulfide bond.

The present invention also provides a pharmaceutical composition comprising an antibody for use in treating cancer, comprising:
a) a heavy chain variable region having the amino acid sequence of SEQ ID NO:13; and
b) a light chain variable region having the amino acid sequence of SEQ ID NO:14,

and an acceptable carrier, diluent, or excipient.

The present invention also provides a pharmaceutical composition comprising an antibody for use in treating cancer, comprising:

a) a heavy chain having the amino acid sequence of SEQ ID NO:21; and

b) a light chain having the amino acid sequence of SEQ ID NO:22,

and an acceptable carrier, diluent, or excipient.

In one embodiment, the antibody is a human IgG1 engineered to reduce the binding of the antibody to an Fc gamma receptor.

The present invention also provides a pharmaceutical composition comprising an antibody for use in treating cancer, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. The present invention further provides a pharmaceutical composition comprising an antibody for use in treating lung cancer, wherein the lung cancer is non-small cell lung cancer or small cell lung cancer. The present invention further provides a pharmaceutical composition comprising an antibody for use in treating breast cancer, wherein the breast cancer is triple-negative breast cancer.

In one embodiment, the composition is administered in simultaneous, separate, or sequential combination with ionizing radiation. In another embodiment, the pharmaceutical composition is administered in simultaneous, separate, or sequential combination with one or more chemotherapeutic agents. In another embodiment, the pharmaceutical composition is administered in simultaneous, separate, or sequential combination with ionizing radiation and one or more chemotherapeutic agents.

The present invention also provides the use of an antibody of the present invention in the manufacture of a medicament for treating cancer, wherein the antibody binds to human TIGIT (SEQ ID NO:31), or to a human TIGIT extracellular domain, e.g., SEQ ID NO: 32, comprising:
a) a heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3; and
b) a light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6.

In one embodiment, the heavy chain of the antibody forms at least one disulfide bond with the light chain of the antibody, and the two heavy chains of the antibody form at least one disulfide bond.

The present invention also provides the use of an antibody of the present invention in the manufacture of a medicament for treating cancer, comprising:
a) a heavy chain variable region having the amino acid sequence of SEQ ID NO:13; and
b) a light chain variable region having the amino acid sequence of SEQ ID NO:14.

The present invention also provides the use of an antibody of the present invention in the manufacture of a medicament for treating cancer, comprising:

a) a heavy chain having the amino acid sequence of SEQ ID NO:21; and

b) a light chain having the amino acid sequence of SEQ ID NO:22.

In one embodiment, the antibody is a human IgG1 engineered to reduce the binding of the antibody to an Fc gamma receptor.

The present invention also provides the use of an antibody of the present invention in the manufacture of a medicament for treating cancer, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. The present invention further provides the use of an antibody of the present invention in the manufacture of a medicament for treating lung cancer, wherein the lung cancer is non-small cell lung cancer or small cell lung cancer. The present invention further provides the use of an antibody of the present invention in the manufacture of a medicament for treating breast cancer, wherein the breast cancer is triple-negative breast cancer.

In one embodiment, the antibody is administered in simultaneous, separate, or sequential combination with ionizing radiation. In another embodiment, the antibody is administered in simultaneous, separate, or sequential combination with one or more chemotherapeutic agents. In another embodiment, the antibody is administered in simultaneous, separate, or sequential combination with ionizing radiation and one or more chemotherapeutic agents.

In embodiments that refer to a method of treatment as described herein, such embodiments are also further embodiments for use in that treatment, or alternatively for the use in the manufacture of a medicament for use in that treatment.

Non-limiting examples of useful chemotherapeutic agents include 5-fluorouracil, hydroxyurea, gemcitabine, pemetrexed, methotrexate, doxorubicin, etoposide, carboplatin, cisplatin, cyclophosphamide, melphalan, dacarbazine, taxol, camptothecin, FOLFIRI, FOLFOX, docetaxel, daunorubicin, paclitaxel, oxaliplatin, and combinations thereof.

The antibodies of the present invention, or pharmaceutical compositions comprising the same, may be administered by parenteral routes, a non-limiting example of which is intravenous administration. The antibodies of the present invention may be administered to a human patient alone with pharmaceutically acceptable carriers, diluents, or excipients in single or multiple doses. A pharmaceutical composition of the present invention may be prepared by methods known in the art (e.g., Remington: The Science and Practice of Pharmacy, 22^{nd} ed. (2012), A. Loyd et al., Pharmaceutical Press).

In one embodiment, the polypeptide molecule of the invention is sterile. In another embodiment, the polypeptide molecule of the invention is substantially pure. In another embodiment, the polypeptide molecule of the invention is substantially pure and sterile.

The bispecific antibodies of the present invention are heterodimeric in that each arm of the antibody exhibits selective monovalent binding to its cognate antigen due in part to the two different heavy chains and the two different light chains. In the present invention, one arm of the bispecific antibody binds human PD-1 (SEQ ID NO:29), or a human PD-1 extracellular domain (ECD), e.g., an ECD-His expression product (SEQ ID NO: 30), while the other arm binds human TIGIT (SEQ ID NO:31), or a TIGIT ECD, e.g., an ECD-His expression product (SEQ ID NO: 32). In a preferred embodiment, one arm of the antibody antagonizes human PD-1 (SEQ ID NO:29), and the other arm antagonizes human TIGIT (SEQ ID NO:31).

The present invention also provides an antibody that binds to human PD-1 (SEQ ID NO:29), or to a PD-1 extracellular domain, e.g., SEQ ID NO: 30, and binds to human TIGIT (SEQ ID NO:31), or to a TIGIT extracellular domain, e.g., SEQ ID NO: 32, comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12.

The present invention also provides an antibody comprising:
a) a first heavy chain variable region having the amino acid sequence of SEQ ID NO:13;
b) to first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18.

The present invention also provides an antibody comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24.

The present invention also provides an antibody (referred to herein as Antibody A) having:

a) a first heavy chain having the amino acid sequence of SEQ ID NO:21;

b) ae first light chain having the amino acid sequence of SEQ ID NO:22;

c) a second heavy chain having the amino acid sequence of SEQ ID NO:23; and

d) a second light chain having the amino acid sequence of SEQ ID NO:24.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody.

In another embodiment, the antibody is a human IgG1 engineered to reduce the binding of the antibody to an Fc gamma receptor.

The present invention also provides a DNA molecule comprising a polynucleotide encoding for at least one polypeptide having the amino acid sequence of SEQ ID NO:21, the amino acid sequence of SEQ ID NO:22, the amino acid sequence of SEQ ID NO:23, and the amino acid sequence of SEQ ID NO:24. In a preferred embodiment, the DNA molecule comprises a polynucleotide comprising at least one of SEQ ID NO: 25, SEQ ID NO: 26, SEQ ID NO: 27 and SEQ ID NO: 28.

The present invention also provides a mammalian cell comprising a DNA molecule comprising a polynucleotide encoding for at least one polypeptide having the amino acid sequence of SEQ ID NO:21, the amino acid sequence of SEQ ID NO:22, the amino acid sequence of SEQ ID NO:23, and the amino acid sequence of SEQ ID NO:24.

The present invention also provides a process for producing an antibody comprising cultivating a mammalian cell capable of expressing the antibody and recovering the antibody, comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody.

The present invention also provides a process for producing an antibody comprising cultivating a mammalian cell capable of expressing the antibody and recovering the antibody, comprising:
a) a first heavy chain variable region having the amino acid sequence of SEQ ID NO:13;
b) a first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18.

The present invention also provides a process for producing an antibody comprising cultivating a mammalian cell capable of expressing the antibody and recovering the antibody, comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24.

The present invention also provides a process for producing an antibody comprising cultivating a mammalian cell capable of expressing the antibody and recovering the antibody; wherein the antibody is a human IgG1 engineered to reduce the binding of the antibody to an Fc gamma receptor.

The present invention also provides an antibody produced by a process comprising cultivating a mammalian cell capable of expressing the antibody and recovering the antibody, the antibody comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody.

The present invention also provides an antibody produced by a process comprising cultivating a mammalian cell capable of expressing the antibody and recovering the antibody, comprising:
a) a first heavy chain variable region having the amino acid sequence of SEQ ID NO:13;
b) a first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18.

The present invention also provides an antibody produced by a process comprising cultivating a mammalian cell capable of expressing the antibody and recovering the antibody, comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24.

The present invention also provides a pharmaceutical composition comprising an antibody, wherein the antibody binds to human PD-1 (SEQ ID NO:29), or to a human PD-1 extracellular domain, e.g., SEQ ID NO: 30, and binds to human TIGIT (SEQ ID NO:31), or to a human TIGIT extracellular domain, e.g., SEQ ID NO: 32, comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12,

and an acceptable carrier, diluent, or excipient.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody.

The present invention also provides a pharmaceutical composition comprising an antibody comprising:
a) a first heavy chain variable region having the amino acid sequence of SEQ ID NO:13;
b) a first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18,

and an acceptable carrier, diluent, or excipient.

The present invention also provides a pharmaceutical composition comprising an antibody comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24, and an acceptable carrier, diluent, or excipient.

In one embodiment, antibody is a human IgG1 engineered to reduce the binding of the antibody to an Fc gamma receptor.

The present invention also provides a method of treating cancer comprising administering to a human patient in need thereof, an effective amount of an antibody, wherein the antibody binds to human PD-1 (SEQ ID NO:29), or a human PD-1 extracellular domain, e.g., SEQ ID NO: 30, and binds to human TIGIT (SEQ ID NO:31), or a human TIGIT extracellular domain, e.g., SEQ ID NO: 32, comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody.

The present invention also provides a method of treating cancer comprising administering to a human patient in need thereof, an effective amount of an antibody, comprising:
a) a first heavy chain variable region having the amino acid sequence of SEQ ID NO:13;
b) a first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18.

The present invention also provides a method of treating cancer comprising administering to a human patient in need thereof, an effective amount of an antibody, comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24.

The present invention also provides methods of treatment and methods for use.

In one embodiment, the antibody is a human IgG1 engineered to reduce the binding of the antibody to an Fc gamma receptor.

The present invention also provides a method of treating cancer comprising administering to a human patient in need thereof, an effective amount of an antibody described herein, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma.

The present invention also provides a method of treating cancer comprising administering to a human patient in need thereof, an effective amount of an antibody described herein, wherein the cancer is non-small cell lung cancer, or small cell lung cancer. The present invention further provides a method of treating cancer comprising administering to a human patient in need thereof, an effective amount of an antibody described herein, wherein the cancer is triple negative breast cancer.

The present invention also provides a method of treating cancer comprising administering to a human patient in need thereof, an effective amount of an antibody described herein, wherein the antibody is administered in combination with ionizing radiation.

The present invention also provides a method of treating cancer comprising administering to a human patient in need thereof, an effective amount of an antibody described herein, wherein the antibody is administered in combination with one or more chemotherapeutic agents.

The present invention also provides a method of treating cancer comprising administering to a human patient in need thereof, an effective amount of an antibody described herein, wherein the antibody is administered in combination with ionizing radiation and one or more chemotherapeutic agents.

In one embodiment, the present invention also provides a method of treating cancer, comprising administering an effective amount of a bispecific antibody disclosed herein in simultaneous, separate, or sequential combination with one or more anti-tumor agents. Non-limiting examples of anti-tumor agents include ramucirumab, necitumumab, olaratumab, gemcitabine, pemetrexed, galunisertib, abemaciclib, cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), cetuximab, an EGFR inhibitor, a Raf inhibitor, a B-Raf inhibitor, a CDK4/6 inhibitor, a CDK7 inhibitor, an idoleamine 2,3-dioxygenase inhibitor, a TGFβ inhibitor, a TGFβ receptor inhibitor, IL-10, and pegylated IL-10 (e.g., pegilodecakin).

The present invention also provides an antibody for use in treating cancer, wherein the antibody binds to human PD-1 (SEQ ID NO:29), or to a human PD-1 extracellular domain, e.g., SEQ ID NO: 30, and binds to human TIGIT (SEQ ID NO:31), or to a human TIGIT extracellular domain, e.g., SEQ ID NO: 32, comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody.

The present invention also provides an antibody for use in treating cancer, comprising:
a) a first heavy chain variable region having the amino acid sequence of SEQ ID NO:13;
b) a first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18.

The present invention also provides an antibody for use in treating cancer, comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24.

In one embodiment, the antibody is a human IgG1 engineered to reduce the binding of the antibody to an Fc gamma receptor.

The present invention also provides an antibody for use in treating cancer, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. The present invention further provides an antibody for use in treating lung cancer, wherein the lung cancer is non-small cell lung cancer or small cell lung cancer. The present invention also provides an antibody for use in treating breast cancer, wherein the breast cancer is triple-negative breast cancer.

In one embodiment, the antibody is administered in simultaneous, separate, or sequential combination with ionizing radiation. In another embodiment, the antibody is administered in simultaneous, separate, or sequential combination with one or more chemotherapeutic agents. In another embodiment, the antibody is administered in simultaneous, separate, or sequential combination with ionizing radiation and one or more chemotherapeutic agents.

The present invention also provides a pharmaceutical composition comprising an antibody for use in treating cancer, wherein the antibody binds to human PD-1 (SEQ ID NO:29), or a human PD-1 extracellular domain, e.g., SEQ ID NO: 30, and binds to human TIGIT (SEQ ID NO:31), or a human TIGIT extracellular domain, e.g., SEQ ID NO: 32, comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12,

and an acceptable carrier, diluent, or excipient.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody

The present invention also provides a pharmaceutical composition comprising an antibody for use in treating cancer, comprising:
a) a first heavy chain variable region having the amino acid sequence of SEQ ID NO:13;
b) a first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18,

and an acceptable carrier, diluent, or excipient.

The present invention also provides a pharmaceutical composition comprising an antibody for use in treating cancer, comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24, and an acceptable carrier, diluent or excipient.

In one embodiment, the antibody is a human IgG1 engineered to reduce the binding of the antibody to an Fc gamma receptor.

The present invention also provides a pharmaceutical composition comprising an antibody for use in treating cancer, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. The present invention further provides a pharmaceutical composition comprising an antibody for use in treating lung cancer, wherein the lung cancer is non-small cell lung cancer or small cell lung cancer. The present invention further provides a pharmaceutical composition comprising an antibody for use in treating breast cancer, wherein the breast cancer is triple-negative breast cancer.

In one embodiment, the composition is administered in simultaneous, separate, or sequential combination with ionizing radiation. In another embodiment, the pharmaceutical composition is administered in simultaneous, separate, or sequential combination with one or more chemotherapeutic agents. In another embodiment, the pharmaceutical composition is administered in simultaneous, separate, or sequential combination with ionizing radiation and one or more chemotherapeutic agents.

The present invention also provides the use of an antibody of the present invention in the manufacture of a medicament for treating cancer, wherein the antibody binds to human PD-1 (SEQ ID NO:29), or to a human PD-1 extracellular domain, e.g., SEQ ID NO: 30, and binds to human TIGIT (SEQ ID NO:31), or to a human TIGIT extracellular domain, e.g., SEQ ID NO: 32, comprising:
a) a first heavy chain comprising an HCDR1 having the amino acid sequence of SEQ ID NO:1, an HCDR2 having the amino acid sequence of SEQ ID NO:2, and an HCDR3 having the amino acid sequence of SEQ ID NO:3;
b) a first light chain comprising an LCDR1 having the amino acid sequence of SEQ ID NO:4, an LCDR2 having the amino acid sequence of SEQ ID NO:5, and an LCDR3 having the amino acid sequence of SEQ ID NO:6;
c) a second heavy chain comprising an HCDR1 having the amino acid sequence of SEQ ID NO:7, an HCDR2 having the amino acid sequence of SEQ ID NO:8, and an HCDR3 having the amino acid sequence of SEQ ID NO:9; and
d) a second light chain comprising an LCDR1 having the amino acid sequence of SEQ ID NO:10, an LCDR2 having the amino acid sequence of SEQ ID NO:11, and an LCDR3 having the amino acid sequence of SEQ ID NO:12.

In one embodiment, the first heavy chain of the antibody forms at least one disulfide bond with the first light chain of the antibody, the second heavy chain of the antibody forms at least one disulfide bond with the second light chain of the antibody, and the first heavy chain of the antibody forms at least one disulfide bond with the second heavy chain of the antibody.

The present invention also provides the use of an antibody of the present invention in the manufacture of a medicament for treating cancer, comprising:
a) a first heavy chain variable region having the amino acid sequence of SEQ ID NO:13;
b) a first light chain variable region having the amino acid sequence of SEQ ID NO:14;
c) a second heavy chain variable region having the amino acid sequence of SEQ ID NO:17; and
d) a second light chain variable region having the amino acid sequence of SEQ ID NO:18.

The present invention also provides the use of an antibody of the present invention in the manufacture of a medicament for treating cancer, comprising:

a) a first heavy chain having the amino acid sequence of SEQ ID NO:21;

b) a first light chain having the amino acid sequence of SEQ ID NO:22;

c) a second heavy chain having the amino acid sequence of SEQ ID NO:23; and

d) a second light chain having the amino acid sequence of SEQ ID NO:24.

In one embodiment, the antibody is a human IgG1 engineered to reduce the binding of the antibody to an Fc gamma receptor.

The present invention also provides the use of an antibody of the present invention in the manufacture of a medicament for treating cancer, wherein the cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma. The present invention further provides the use of an antibody of the present invention in the manufacture of a medicament for treating lung cancer, wherein the lung cancer is non-small cell lung cancer or small cell lung cancer. The present invention further provides the use of an antibody of the present invention in the manufacture of a medicament for treating breast cancer, wherein the breast cancer is triple-negative breast cancer.

In one embodiment, the antibody is administered in simultaneous, separate, or sequential combination with ionizing radiation. In another embodiment, the antibody is administered in simultaneous, separate, or sequential combination with one or more chemotherapeutic agents. In another embodiment, the antibody is administered in simultaneous, separate, or sequential combination with ionizing radiation and one or more chemotherapeutic agents.

In embodiments that refer to a method of treatment as described herein, such embodiments are also further embodiments for use in that treatment, or alternatively for the use in the manufacture of a medicament for use in that treatment.

Non-limiting examples of useful chemotherapeutic agents include 5-fluorouracil, hydroxyurea, gemcitabine, pemetrexed, methotrexate, doxorubicin, etoposide, carboplatin, cisplatin, cyclophosphamide, melphalan, dacarbazine, taxol, camptothecin, FOLFIRI, FOLFOX, docetaxel, daunorubicin, paclitaxel, oxaliplatin, and combinations thereof.

The antibodies of the present invention, or pharmaceutical compositions comprising the same, may be administered by parenteral routes, a non-limiting example of which is intravenous administration. The antibodies of the present invention may be administered to a human patient alone with pharmaceutically acceptable carriers, diluents, or excipients in single or multiple doses. A pharmaceutical composition of the present invention may be prepared by methods known in the art (e.g., Remington: The Science and Practice of Pharmacy, 22^{nd} ed. (2012), A. Loyd et al., Pharmaceutical Press).

In one embodiment, the polypeptide molecule of the invention is sterile. In another embodiment, the polypeptide molecule of the invention is substantially pure. In another embodiment, the polypeptide molecule of the invention is substantially pure and sterile.

Dosage regimens for administering a polypeptide molecule of the invention may be adjusted to provide the optimum desired response (e.g., a therapeutic effect).

In one embodiment, when a polypeptide molecule of the invention binds to human TIGIT or, it antagonizes human TIGIT. In another embodiment, when a polypeptide molecule of the invention binds to human PD-1, it antagonizes human PD-1. As used herein, the term “antagonize” refers to the act of blocking, interrupting, suppressing, inhibiting or reducing a biological activity of interest. In this regard, the polypeptide molecules, e.g., antibodies, of the present invention antagonize human PD-1 by binding to human PD-1 and blocking the binding of human PD-L1 to human PD-1, and antagonize human TIGIT by binding to human TIGIT and blocking the binding of human TIGIT to CD155 and or to CD112.

The term “antibody” as used herein refers to a monomeric or dimeric immunoglobulin molecule having a heavy chain and a light chain that recognizes and binds to a target, such as a protein, peptide or polypeptide. In one embodiment, the antibody specifically binds to the target. Each heavy chain is comprised of an N-terminal HCVR (heavy chain variable region) and an HCCR (heavy chain constant region). Each light chain is comprised of an N-terminal LCVR (light chain variable region) and a LCCR (light chain constant region). The constant region of the heavy chains contain CH1, CH2, and CH3 domains.

The term “antibody fragment” is a fragment of an antibody that retains the ability to bind to the target to which the intact antibody binds. In one embodiment, the antibody fragment specifically binds to the target. In another embodiment, the antibody fragment comprises HCDRs 1-3 and LCDRs 1-3 of the intact antibody. In another embodiment, the antibody fragment comprises the HCVR and LCVR of the intact antibody.

Unless otherwise indicated herein, “TIGIT” refers to human TIGIT, and “PD-1” refers to human PD-1.

The term “binds” as used herein refers to the molecular interaction between two molecules, e.g., a polypeptide molecule of the invention and TIGIT, PD-1, or TIGIT and PD-1. The term “monospecific binding” refers to binding to one target, e.g., human TIGIT or human PD-1. The term “bispecific binding” refers to binding to human TIGIT and to human PD-1. The term “polyspecific binding” refers to binding to human TIGIT, human PD-1 and ono or two other targets.

The terms “selectively binds” or “specifically binds” mean that a polypeptide molecule of the invention interacts more frequently, more rapidly, with greater duration, with greater affinity, or with some combination of the above to human TIGIT, or to PD-1 or to human TIGIT and human PD-1, than do other substances. In one embodiment, “specifically binds” means that a polypeptide molecule of the invention binds to human TIGIT, or to human PD-1 or to human TIGIT and human PD-1 with a K_{D} of about 0.1 mM or less. In another embodiment, “specifically binds” means that a polypeptide molecule of the invention binds to human TIGIT, or to human PD-1 or to human TIGIT and human PD-1 with a K_{D} of about 0.01 mM or less. In another embodiment, “specifically binds” means that a polypeptide molecule of the invention binds to human TIGIT, or to human PD-1 or to human TIGIT and human PD-1 with a K_{D} of about 0.001 mM or less. In another embodiment, “specifically binds” means that a polypeptide molecule of the invention binds to human TIGIT, or to human PD-1 or to human TIGIT and human PD-1 with a K_{D} of about 0.0001 mM or less. In another embodiment, the polypeptide molecule of the invention binds to human TIGIT with a K_{D} that is different than the K_{D} with which the polypeptide molecule binds to human PD-1. In another embodiment, the polypeptide molecule binds to human TIGIT about 10-fold more tightly than it binds to human PD-1.

In one embodiment, the term “polypeptide molecule” as used herein refers to a molecule that comprises a polymer of amino acid residues. In another embodiment, the polypeptide molecule consists of a polymer of amino acid residues.

In one embodiment, the polypeptide molecule is an scFv molecule that binds to human TIGIT, or to human PD-1, or to human TIGIT and human PD-1. In another embodiment, the scFv molecule binds specifically to human TIGIT, or to human PD-1, or to human TIGIT and human PD-1. The scFv molecule can be monospecific (binds to human TIGIT or human PD-1), bispecific (binds to human TIGIT and human PD-1), or polyspecific (binds to human PD-1, human TIGIT and/or another target).

In one embodiment, the polypeptide molecule is an antibody that binds to human TIGIT, or to human PD-1, or to human TIGIT and human PD-1. In another embodiment, the antibody binds specifically to human TIGIT, or to human PD-1, or to human TIGIT and human PD-1. The antibody can be monospecific (binds to human TIGIT or human PD-1), bispecific (binds to human TIGIT and human PD-1), or polyspecific (binds to human PD-1, human TIGIT and to one or two other targets).

In one embodiment, the polypeptide molecule is an antibody fragment that binds to human TIGIT, or to human PD-1, or to human TIGIT and human PD-1. In another embodiment, the antibody fragment binds specifically to human TIGIT, or to human PD-1, or to human TIGIT and human PD-1. The antibody fragment can be monospecific (binds to human TIGIT or human PD-1), bispecific (binds to human TIGIT and human PD-1), or polyspecific (binds to human PD-1, human TIGIT and to one or two other targets).

The term “substantially pure” as used herein refers to material, e.g., a polypeptide molecule of the invention, that is at least 50%, 60%, 70%, 80%, 90%, 95%, 96%, 97%, 98%, 99% or 100% free of contaminants.

Synonyms for “TIGIT” are WUCAM, Vstm3, and VSIG9.

Synonyms for “CD155” are poliovirus receptor, PVR, Nec1-5, NECL5, Tage4, HVED and PVS.

Synonyms for “CD112” are Nectin cell adhesion molecule 2, nectin-2, NECTIN2, PRR-2, PVRL2, PVRR2 and HVEB.

Synonyms for “CD226” are DNAX accessory molecule-1, DNAM-1, DNAM1, PTA1 and TLiSA1.

The term “treating” (or “treat” or “treatment”) refers to slowing, interrupting, arresting, alleviating, stopping, reducing, or reversing the progression or severity of an existing symptom, disorder, condition, or disease.

The term “effective amount” means the amount of a polypeptide molecule of the present invention or a pharmaceutical composition comprising an antibody of the present invention that elicits the biological or medical response or desired therapeutic effect on a tissue, system, animal, mammal or human that is being sought by the researcher, medical doctor, or other clinician. An effective amount of the polypeptide molecule may vary according to factors such as the disease state, age, sex, and weight of the individual, and the ability of the polypeptide molecule to elicit a desired response in the individual. An effective amount is also one in which any toxic or detrimental effect of the antibody is outweighed by the therapeutically beneficial effects.

An isolated DNA molecule encoding a HCVR region may be converted to a full-length heavy chain gene by operably linking the HCVR-encoding DNA to another DNA molecule encoding heavy chain constant regions. The sequences of human, as well as other mammalian, heavy chain constant region genes are known in the art. DNA fragments encompassing these regions may be obtained, e.g., by standard PCR amplification.

An isolated DNA molecule encoding a LCVR region may be converted to a full-length light chain gene by operably linking the LCVR-encoding DNA to another DNA molecule encoding a light chain constant region. The sequences of human, as well as other mammalian, light chain constant region genes are known in the art. DNA fragments encompassing these regions may be obtained by standard PCR amplification.

As used herein, the term “CDR” refers to an antibody complementarity determining region, the term “HCDR” refers to an antibody heavy chain CDR, and the term “LCDR” refers to an antibody light chain CDR. For the purposes of the present invention, the North CDR definitions are used. The North CDR definition (North et al., “A New Clustering of Antibody CDR Loop Conformations”, Journal of Molecular Biology, 406, 228-256 (2011)) is based on affinity propagation clustering with a large number of crystal structures.

The term “modified human IgG1” as used herein means a human IgG1 engineered to reduce the binding of the human IgG1 to at least one human Fc gamma receptor. Typically this is performed by mutating residues that lead to a reduction in the binding of the antibody to the Fc gamma receptor(s), e.g., P329A, L234A and L235 A mutations.

The term “solid tumor” refers to a tumor in a tissue that is not blood, lymphatics or bone marrow.

Methods for assaying TIGIT activity in vitro are known to those of ordinary skill in the art, for example in He et al., Cancer Res 2017; 77: 6375-6388; Yu et al., Nature Immunology 2009; 10(1): 48-57; Johnston et al., Cancer Cell 2014; 26: 923-937; Stanietskya, et al., PNAS 2009; 106(42): 17858-17863; Lozano et al., J Immunol. 2012; 188(8): 3869-3875.

Methods for assaying PD-1 activity in vitro are known to those of ordinary skill in the art, for example in Carpenito et al., J Immunother Cancer 2018; 6(1):31; Ghosh et al., Mol Cancer Ther. 2019; 18(3):632-641; Stewart et al., Cancer Immunol Res. 2015; 3(9):1052-62; Maute et al., PNAS 2015; 112(47): E6506-14.

In vivo murine models of solid tumor are well known to those of ordinary skill in the art, as shown herein, and as disclosed, e.g., in Sanmamed M F, et al., Ann. Oncol. 2016; 27: 1190-1198; Manning H C, et al., J. Nucl. Med 2016; 57(Suppl. 1): 60S-68S; Teich B A. Cancer Ther. 2006; 5: 2435; Rongvaux A, et al., Ann. Rev. Immunol. 2013; 31: 635-74; Stylli S S, et al., J. Clin. Neurosci 2015; 619-26; Oh T, et al., J. Transl. Med. 2014; 12: 107-117; Newcomb, E W, et al., Radiation Res. 2010; 173: 426-432; Song Y, et al., Proc Natl. Acad. Sci. USA 2013; 110: 17933-8; and Rutter E M, et al., Scientific Reports 2017; 7: DOI: 10.1038/s41598-017-02462-0.

A DNA molecule of the present invention is a DNA molecule that comprises a non-naturally occurring polynucleotide sequence encoding a polypeptide having the amino acid sequence of at least one of the polypeptides in an antibody of the present invention.

The polynucleotides of the present invention may be expressed in a host cell after the sequences are operably linked to an expression control sequence. The expression vectors are typically replicable in the host organisms either as episomes or as an integral part of the host chromosomal DNA. Commonly, expression vectors contain selection markers, e.g., tetracycline, neomycin, and dihydrofolate reductase, to permit detection of those cells transformed with the desired DNA sequences.

An expression vector containing the polynucleotide sequences of interest (e.g., the polynucleotides encoding the polypeptides of a polypeptide molecule and expression control sequences) can be transferred into a host cell by known methods, which vary depending on the type of host cells.

A polypeptide molecule of the present invention may readily be produced in mammalian host cells, non-limiting examples of which includes CHO, NS0, HEK293 or COS cells. The host cells may be cultured using techniques known in the art.

Various methods of protein purification may be employed to purify an antibody of the present invention and such methods are known in the art and described, for example, in Deutscher, Methods in Enzymology 182: 83-89 (1990) and Scopes, Protein Purification: Principles and Practice, 3rd Edition, Springer, N.Y. (1994).

Sequences referred to herein are numbered according to the sequence identifier numbers listed in Table 1.

TABLE 1
Sequence identifier numbers

| | Anti-human | Anti-human |
|---|---|---|
| | TIGIT Arm | PD-1 Arm |
| HCDR1 | 1 | 7 |
| HCDR2 | 2 | 8 |
| HCDR3 | 3 | 9 |
| LCDR1 | 4 | 10 |
| LCDR2 | 5 | 11 |
| LCDR3 | 6 | 12 |
| HCVR | 13 | 17 |
| LCVR | 14 | 18 |
| HCCR | 15 | 19 |
| LCCR | 16 | 20 |
| Heavy chain | 21 | 23 |
| Light chain | 22 | 24 |
| DNA Heavy Chain | 25 | 27 |
| DNA Light Chain | 26 | 28 |

| | |
|---|---|
| Human PD-1 | 29 |
| Human PD-1 ECD-His | 30 |
| Human TIGIT | 31 |
| Human TIGIT ECD-His | 32 |

HCCR: Heavy chain constant region;
LCCR: Light chain constant region;
HCVR: Heavy chain variable region;
LCVR: Light chain variable region;
ECD: extracellular domain

### EXAMPLES

### Antibody A Expression and Purification

The antibodies of the present invention may be expressed and purified essentially as follows. An appropriate host cell, such as HEK 293 or CHO, may be either transiently or stably transfected with an expression system for secreting antibodies using an optimal predetermined heavy chain:light chain vector ratio or a single vector system encoding both heavy chain and light chain. Antibody A of the present invention may be either transiently or stably transfected with an expression system for secreting antibodies using one or more DNA molecules encoding for a first heavy chain having the amino acid sequence of SEQ ID NO:21, a first light chain having the amino acid sequence of SEQ ID NO:22, a second heavy chain having the amino acid sequence of SEQ ID NO:23 and a second light chain having the amino acid sequence of SEQ ID NO:24.

The antibodies may be purified using one of many commonly-used techniques. For example, the medium may be conveniently applied to a Mab Select column (GE Healthcare), or KappaSelect column (GE Healthcare), that has been equilibrated with a compatible buffer, such as phosphate buffered saline (pH 7.4). The column may be washed to remove nonspecific binding components. The bound antibody may be eluted, for example, by pH gradient (such as 20 mM Tris buffer pH 7.0 to 10 mM sodium citrate buffer pH 3.0, or phosphate buffered saline pH 7.4 to 100 mM glycine buffer pH 3.0). Antibody fractions may be detected, such as by UV absorbance or SDS-PAGE, and then may be pooled. Further purification is optional, depending on the intended use. The purified antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purified antibody may be immediately frozen at −70° C. or may be lyophilized.

### Antibody A Binds to Human PD-1 and Human TIGIT

A Biacore® T200 (GE Healthcare, Piscataway, N.J.) is used to measure the binding kinetics and affinities of Antibody A to soluble human PD-1 extracellular domain (ECD) (Sino Biologicals, Cat #10377-H08H) and human TIGIT-ECD by surface plasmon resonance at 37° C. Samples are diluted in HBS-EP+ (10 mM HEPES, 150 mM NaCl, 0.05% Tween-20, pH 7.6) running buffer (Teknova Cat #H8022). Protein A CM5 S Series Sensor chip (GE Healthcare Cat #29127555) was purchased from GE Healthcare.

Binding was evaluated using multi-cycle kinetics by an antibody capture method. Each cycle was performed at 37° C. at a flow rate of 10 μL/min for antibody capture to the Protein A chip and 100 μL/min for analyte association and dissociation. Each cycle consists of the following steps: injection of Antibody A at 2 μg/mL in HBS-EP+ targeting Rmax values of 50 RU on flow cell, injection of 180 or 200-seconds of analyte in HBS-EP+ (concentration range of 1000 nM to 1.95 nM by two-fold serial dilution for PD-1-ECD-His (human PD1-ECD-his (Sino Biologicals, Cat: 10377-H08H) and human TIGIT-ECD-His (SEQ ID NO: 32), respectively) followed by 600-second dissociation phase, and regeneration using 5 μL of 10 mM glycine hydrochloride, pH 1.5 over a 30-second contact time utilizing a 10 μL/min flow rate. All analyte concentrations were determined utilizing monomeric molecular weight (MW) values. Association rates (kₒₙ) and dissociation rates (k_{off}) for human PD-1-ECD were evaluated using double referencing by flow-cell 1 reference subtraction in addition to 0 nM blank subtraction and fit to “1:1 (Langmuir) binding” model in the BIAevaluation software version 4.1. The dissociation constant (K_{D}) was calculated from the binding kinetics according to the relationship K_{D}=K_{off}/Kₒₙ. Stoichiometry=[RUₘₐₓ/RU_{captured}]/[MW_{analyte}/MW_{antibody}] where MW_{AntibodyA} is 150 kDa. Values are reported as mean±standard deviation.

In experiments performed essentially as described above, the results in Table 2 demonstrate that Antibody A binds to human PD-1-ECD, human TIGIT-ECD, cynomolgus PD-1 and cynomolgus TIGIT.

TABLE 2

| | On Rate (kₒₙ) | Off Rate (k_{off}) | Affinity (K_{D}) | Stoichiometry |
|---|---|---|---|---|
| Species | (M⁻¹s⁻¹) (±SE) | (s⁻¹) (±SE) | (M)^{a} (±SE) | (±SE) |
| Human PD-1 | 2.0 ± 0.2 × 10⁵ | 5.0 ± 0.7 × 10⁻⁴ | 2.43 ± 0.04 × 10⁻⁹ | 1.29 ± 0.02 |
| ECD (n = 3) | | | | |
| Cynomolgus | 1.8 ± 0.2 × 10⁵ | 4.5 ± 0.7 × 10⁻⁴ | 2.43 ± 0.14 × 10⁻⁹ | 1.08 ± 0.01 |
| PD-1 (n = 3) | | | | |
| Human TIGIT | 1.6 ± 0.3 × 10⁶ | 4.2 ± 1.2 × 10⁻⁴ | 0.25 ± 0.04 × 10⁻⁹ | 0.837 ± 0.003 |
| ECD (n = 3) | | | | |
| Cynomolgus | 2.9 ± 0.4 × 10⁶ | 1.1 ± 0.1 × 10⁻³ | 0.36 ± 0.01 × 10⁻⁹ | 0.90 ± 0.01 |
| TIGIT (n = 3) | | | | |

### Antibody A Antagonizes Human PD-1/PD-L1 Activity in a Cell Based Assay.

The ability of Antibody A to antagonize the activity mediated by human PD-1 binding to human PD-L1 is tested using an NFAT-Luc reporter assay. Briefly, CHO-K1 cells expressing human PD-L1 and an artificial cell surface T cell receptor (TCR) activator (Promega CS187108, part of PD-1/PD-L1 Blockade Assay System, Propagation Model CS187109) are used as antigen presenting cells. Human TIGIT is introduced by retroviral transfer into Jurkat cells expressing human PD-1 and an NFAT-Luc2 reporter (GloResponse NFAT-luc2/PD-1 Jurkat, Promega CS187102, part of PD-1/PD-L1 Blockade Assay System, Propagation Model CS187109). CHO-K1+PD-L1+PVR+TCR activator cells (at passages 7-9) are detached with trypsin and seeded at 40,000 cells/well in white opaque 96-well tissue culture plates (Costar 35-3296) in 100 ul of growth medium. CHO-K1+PD-L1+TCR activator growth medium consists of Ham's F-12 medium (Corning Cellgro 10-080-CV) with 10% defined FBS (HyClone SH30070.03), 200 μg/mL hygromycin B (Thermo Fisher 10687-010), and 250 μg/mL G418 (Geneticin, Corning 30-234-CI). Cells are grown overnight at 37° C., 5% CO₂, and 95% RH. On the following day, antibodies as shown in Table 3 are prepared with 2× working concentration in RPMI 1640 with 2 mM L-glutamine and 10 mM HEPES (Gibco 22400) with 2% defined FBS (HyClone SH30070.03).

Jurkat cells expressing human PD-1, human TIGIT, and an NFAT-Luc2 reporter are propagated in RPMI 1640 with 2 mM L-glutamine and 10 mM HEPES (Gibco), 10% defined FBS (HyClone), 100 μg/ml hygromycin B (Thermo Fisher), 500 μg/mL G418 (Geneticin, Corning), and 1 μg/mL puromycin (Calbiochem 540411, in sterile water). Jurkat cells between passages 5 to 7 are centrifuged, and resuspended in RPMI/2% defined FBS at a concentration of 1.25×10⁶ cells/mL. 95 μl of media is carefully removed from the monolayers of CHO+PD-L1+PVR+TCR activator cells in the 96-well plates. 40 μl of 2× concentration antibodies as prepared above (including medium alone control) are added in triplicates for each treatment as indicated in Table 3. Then, 40 μl of the resuspended Jurkat+PD-1+TIGIT+NFAT-Luc2 cells are added per well (50,000 cells/well). Assay plates are incubated for 6 hrs at 37° C., 5% CO₂, 95% RH. Plates are equilibrated for 5 to 10 minutes at room temperature (RT) at the end of incubation. 80 μL/well of reconstituted Bio-Glo™ luciferase substrate (Promega G7940) is added and plates are further incubated for 5-10 minutes at RT. Plates are read on a Perkin Elmer Envision Multimode Reader, with EnVision Manager software v.1.13.3009.1409, ultrasensitive mode, and a 0.2 second integration time. Within each plate, luminescence values (relative light unit (RLU)) are normalized to values obtained from cells treated with medium alone (Fold Induction=RLU treatment/RLU medium alone control.). EC₅₀ values are calculated using GraphPad Prism 7 software.

In experiments performed essentially as described above, the results in Table 3 demonstrate that the EC₅₀ values for Antibody A and the anti-human PD-1-IgG4-PAA are 1.838 nM and 1.226 nM, respectively, and that Antibody A binds to and antagonizes human PD-1/human PD-L1 binding in a cell based assay.

TABLE 3

| | | Anti-human | Anti-human | |
|---|---|---|---|---|
| | | TIGIT- | PD-1- | |
| | Antibody A | hIgG1-EN | IgG4-PAA | hIgG1-EN |
| EC50 (nM) | 1.838 | 0.6664 | 1.226 | >171 |
| Max Fold | 2.21 | 1.7 | 1.84 | 1.08 |
| Change (at | | | | |
| 171 nM) | | | | |

### Antibody A Antagonizes Human TIGIT in a Cell Based Assay

Both human PD-1 and TIGIT are expressed or co-expressed in activated tumor infiltrating lymphocytes. The ability of Antibody A to antagonize human TIGIT-mediated activity is tested in Jurkat NFAT-Luc reporter assays, engineered to co-express human PD-1 (9,000 PD-1 receptors/cell) and human TIGIT (5,500 TIGIT receptors/cell). Briefly, antibodies as shown in Table 4 are incubated with Jurkat+human TIGIT+human PD-1+NFAT-Luc cells for 6 hours. Bio-Glo luciferase substrate is added and luminescence is read at the end of incubation. Data (Fold Induction=RLU treatment/RLU medium alone control) are represented as the mean of triplicate wells per treatment in Table 4.

In experiments performed essentially as described above, the results in Table 4 demonstrate that Antibody A binds to and antagonizes human TIGIT in a cell based assay.

TABLE 4

| | | Anti-human | Anti-human | |
|---|---|---|---|---|
| | | PD-1- | TIGIT- | |
| | Antibody A | hIgG4-PAA | hIgG1-EN | hIgG1-EN |
| EC50 (nM) | 7.869 | >171 | 0.1806 | >171 |
| Max Fold | 1.95 | 1.22 | 1.64 | 1.1 |
| Change (at | | | | |
| 171 nM) | | | | |

### Antibody A Binds to PD-1 and TIGIT Simultaneously in a Cell Based Assay

PD-1 and TIGIT receptors are tagged with Prolink and Enzyme Activator respectively and co-expressed in 293 cells. Upon binding of Antibody A to the human PD-1 and human TIGIT receptors, the receptors are brought in close proximity, enabling reconstitution of the active beta-galactosidase enzyme which hydrolyzes the substrate to generate a chemiluminescent signal.

In experiments performed essentially as described above, the results in Table 5 demonstrate that Antibody A physically engages with the human PD-1 and human TIGIT receptors simultaneously. No effect is seen with the control IgG1 or the anti-human TIGIT and anti-human PD-1 antibodies or with the combination of anti-human TIGIT and anti-human PD1 antibodies.

TABLE 5

| | | | Anti-human | |
|---|---|---|---|---|
| | | | PD-1- | |
| | | Anti-human | hIgG4-PAA + | |
| | | PD-1-hIgG4- | Anti-TIGIT- | hIgG1- |
| Species | Antibody A | PAA | hIgG1-EN | EN |
| EC50 (nM) | 0.4307 | >200 | >200 | >200 |

### Antibody A Induces T Cell Activation in a Mixed Leukocyte Reaction (MLR Reaction)

The human PD-1 blocking function of Antibody A is examined in human allo MLR assays. Human PBMCs are obtained either frozen (AllCells) or from fresh whole blood subjected to plasmapheresis (Indiana Blood Center) and separated on a Ficoll-Paque PLUS (GE Healthcare) density gradient. CD14⁺ monocytes are isolated with Human Monocyte Isolation Kit II or CD14 Microbeads (Miltenyi Biotec) and an AutoMACS Pro separator (Miltenyi Biotec). Immature dendritic cells (DCs) are generated by culturing monocytes in complete RPMI-1640 medium containing 10% FBS in the presence of 1,000 IU/mL hGM-CSF (R&D; 215-GM-050, or Sanofi; Leukine, sargramostim; NDC 0024-5843-01) and 500 IU/mL hIL-4 (R&D; 204-IL-050, or another source) for 2 days (Table 6). CD4⁺ T cells are purified from fresh human PBMCs of different healthy donors (AllCells or Indiana Blood Center) using a Human CD4⁺ T Cell Isolation Kit (Miltenyi Biotec). The two types of cells from different donors are then mixed in 96-well V-bottom plates in complete AIM-V medium (Thermo Fisher Scientific) containing 5×10⁴ to 1×10⁵ CD4⁺ T cells and 5×10³ immature DCs per well. Antibodies as shown in Table 6 are serially diluted and added to the plates in triplicates at 100 uL/well. Plates are incubated for 4 days at 37° C. in 5% CO₂. Supernatants are harvested and subjected to a human IFN-γ ELISA (R&D Systems; SIF50, or DY285) according to manufacturer instructions. The antibodies are tested across nine different donor pairs. EC50 values are calculated using data from three T:DC donor pairs, with GraphPad Prism software (GraphPad Software).

In experiments performed essentially as described above, the results in Table 6 surprisingly demonstrate that Antibody A exhibits enhanced human PD-1 blocking activity when compared to the anti-PD-1 antibody alone, or to the anti-human PD-1+anti-human TIGIT combination, as measured by the maximum fold increase in IFNγ levels relative to IgG1 control.

TABLE 6

| | | | | Anti-human |
|---|---|---|---|---|
| | | | | PD-1- |
| | | | | hIgG4-PAA + |
| | | Anti-human | Anti-human | Anti-human |
| | | PD-1- | TIGIT- | TIGIT- |
| Abs | Antibody A | hIgG4-PAA | hIgG1-EN | hIgG1-EN |
| EC50 (nM) | 9.07 | 0.016 | 24.41 | 0.062 |
| IFNγ Max | 12.27 | 3.85 | 2.83 | 5.64 |
| Fold Increase | | | | |

### Antibody A Induces T Cell Activation in a Tetanus Recall Assay

Frozen PBMC from a tetanus toxoid responder is thawed with warm complete AIM-V medium and rested for 24 hours. After resting, cells are passed through a 30 micron filter to remove large debris and aggregates. Cells are counted and resuspended to 2.5×10⁶ cells/mL in complete AIM-V medium and seeded at 5×10⁵ cells/well in 200 uL in a U-bottom 96 well plate. Antibodies as shown in Table 7 are added at 20 ug/ml and serially diluted 1:3. Cells are stimulated with 4 ng/mL tetanus toxoid and incubated at 37° C. for 48 hours. IFNγ levels in the supernatant is then quantified with an MSD kit (Mesoscale Discovery).

In experiments performed essentially as described above, the results in Table Table 7 and Table 8 demonstrate that the addition of Antibody A (Table 7), or anti-human PD-1+anti-human TIGIT combination (Table 8) enhances T cell activation in a dose-dependent manner as measured by IFNγ release.

TABLE 7

| Antibody | Antibody A treated cells | | | Antibody A | Antibody A |
|---|---|---|---|---|---|
| ug/ml | IFNγ levels | | | mean | SD |
| 20 | 4890.53 | 927.51 | 3601.64 | 3139.89 | 2021.46 |
| 6.67 | 4400.90 | 2865.88 | 2901.18 | 3389.32 | 876.23 |
| 2.22 | 3801.46 | 2733.48 | 2775.13 | 3103.36 | 604.93 |
| 0.74 | 1717.98 | 7374.75 | 2090.72 | 3727.82 | 3163.83 |
| 0.25 | 1224.61 | 1771.20 | 2698.75 | 1898.19 | 745.23 |
| 0.08 | 1394.55 | 684.00 | 1493.15 | 1190.56 | 441.46 |

TABLE 8

| | | | | Anti-human PD- | Anti-human |
|---|---|---|---|---|---|
| | Anti-human PD-1-hIgG5- | | | 1-hIgG4-PAA + | PD-1-hIgG4- |
| | PAA + Anti-human TIGIT | | | Anti-human | PAA + Anti- |
| Antibody | hIgG1-EN treated cells | | | TIGIT | human TIGIT |
| ug/ml | IFNγ levels | | | hIgG1-EN mean | hIgG1-EN SD |
| 20 | 3404.44 | 5641.61 | 4724.52 | 4590.19 | 1124.61 |
| 6.67 | 1286.80 | 2718.54 | 2783.72 | 2263.02 | 846.06 |
| 2.22 | 2022.00 | 4312.19 | 14129.19 | 6821.13 | 6431.73 |
| 0.74 | 1259.72 | 1401.27 | 2815.09 | 1825.36 | 860.05 |
| 0.25 | 488.85 | 1132.18 | 2171.95 | 1264.33 | 849.30 |
| 0.08 | 839.55 | 1235.07 | 792.87 | 955.83 | 242.95 |

Antibody A Demonstrates Antitumor Efficacy in the HCC827 NSG Tumor Xenograft Model Engrafted with Human T Cells.

On Day 0, 10×10⁶HCC827 cells are resuspended in 0.2 mL matrigel solution and subcutaneously implanted into the right flank of female NOD/SCID Gamma (NSG) mice (Jackson Laboratories) engrafted with human T cells. On Day 40, mice are randomized at n=8 and dosed intraperitoneally (ip) at 10 mg/kg once a week for 4 weeks per treatment group. Treatment groups include control IgG, Antibody A, Anti-human PD-1-hIgG4-PAA, Anti-human TIGIT-hIgG1-EN and Anti-human PD-1-hIgG4-PAA+Anti-human TIGIT-hIgG1-EN antibodies. Antibody A is also dosed at 1 mg/kg and 3 mg/kg weekly for 4 weeks. Body weight and tumor volume are measured twice a week. Tumor volume (mm³) is calculated as π/6*Length*Width² and % T/C is calculated as 100×ΔT/ΔC, if ΔT>0 of the geometric mean values. Statistical analysis is performed using the procedures in the SAS software.

In experiments performed essentially as described above, the results in Table 9 demonstrate that Antibody A dosed at 1 mg/kg, 3 mg/kg or 10 mg/kg significantly inhibits tumor growth (p<0.001 respectively) in the human T Cell engrafted mice, relative to the control IgG treated group. Surprisingly, Antibody A at all 3 doses also demonstrates statistically significant efficacy when compared to the anti-human PD-1+anti-human TIGIT combination treatment group with p<0.001 and p<0.334 respectively.

TABLE 9

| | | p-value for | |
|---|---|---|---|
| | Xenograft | tumor volume | % T/C |
| Control IgG 10 mg/kg | HCC827 | p = .174 | 122.9 |
| | unengrafted | | |
| Control IgG 10 mg/kg | HCC827 | NA | NA |
| Anti-human PD-1- | HCC827 | p = .872 | 97.3 |
| hIgG4-PAA 10 mg/kg | | | |
| Anti-human Tigit- | HCC827 | p < .001 | 28.4 |
| hIgG1-EN 10 mg/kg | | | |
| Anti-human PD-1- | HCC827 | p = .334 | 85.8 |
| IgG4-PAA + Anti-human | | | |
| TIGIT hIgG1-EN | | | |
| 10 mg/kg each | | | |
| Antibody A 1 mg/kg | HCC827 | p < .001 | 28.4 |
| Antibody A 3 mg/kg | HCC827 | p < .001 | 25.3 |
| Antibody A 10 mg/kg | HCC827 | p < .001 | 24 |

NA = not applicable

### Antibody A Demonstrates Antitumor Efficacy and Increased CD226+ CD8 T Cells and CD226+ NK Cells in the HCC827 NSCLC CD34 NSG Tumor Xenograft Model.

On Day 0, 10×10⁶ HCC827 are subcutaneously implanted into the right flank of female NOD/SCID Gamma (NSG) mice engrafted with CD34+ hematopoietic stem cells (Jackson Laboratories). On Day 21, mice are randomized at n=8 per group and dosed intraperitoneally (ip) at 10 mg/kg once a week for 4 weeks per treatment group. Treatment groups include control IgG, Antibody A, Anti-human PD-1-hIgG4-PAA, anti-human TIGIT-hIgG1-EN and anti-human PD-1-hIgG4-PAA+anti-human TIGIT-hIgG1-EN Antibodies. Body weight and tumor volume are measured twice a week. Tumor volume (mm³) is calculated as n/6*Length*Width² and % T/C is calculated as 100×ΔT/ΔC, if ΔT>0 of the geometric mean values. Statistical analysis is performed using the MIXED procedures in SAS software.

In experiments performed essentially as described above, the results in Table 10 demonstrate that Antibody A dosed at 10 mg/kg significantly inhibits tumor growth (p<0.001) in the human CD34+ hematopoietic stem cell engrafted mice, relative to the control IgG treated group. Surprisingly, Antibody A also demonstrates significant anti-tumor efficacy when compared to the anti-human PD-1+anti-human TIGIT combination treatment group with p<0.001 and p<0.006 respectively.

TABLE 10

| | | p-value for | |
|---|---|---|---|
| | Xenograft | tumor volume | % T/C |
| Control IgG 10 mg/kg | HCC827 | p < 0.001 | 5874.0 |
| | unengrafted | | |
| Control IgG 10 mg/kg | HCC827 + CD34 | NA | NA |
| Anti-human PD-1- | HCC827 + CD34 | p = 0.047 | 74.6 |
| hIgG4-PAA 10 mg/kg | | | |
| Anti-human TIGIT | HCC827 + CD34 | p = 0.040 | 136.5 |
| hIgG1-EN | | | |
| 10 mg/kg | | | |
| Anti-human PD-1- | HCC827 + CD34 | p = 0.006 | 64.6 |
| hIgG4-PAA + Anti-human | | | |
| TIGIT-hIgG1-EN | | | |
| 10 mg/kg each | | | |
| Antibody A 10 mg/kg | HCC827 + CD34 | p < 0.001 | 53.7 |

NA = not applicable

At study termination, tumors are collected and processed into a single cell suspension. Tumor infiltrating lymphocytes (TILs) are stained with antibodies in 300 ul FACS buffer. Flow data is acquired using LSRFortessa X20 and analyzed using a FlowJo 10. CD226+ CD8 T cells are shown in Table 11 as % of total CD8 T cells (CD8+CD3+CD45+ live lymphocytes) in the TILs of each mouse. CD226+ NK cells are shown in Table 11 as % of total NK cells (CD56+CD3−CD45+ live lymphocytes) in the TILs of each mouse.

In experiments performed essentially as described above, the results in Table 11 and Table 12 demonstrate that the Antibody A treated mice exhibit an increase in the percentage of CD226+ CD8 T cells and CD226+ NK cells, whereas the anti-human PD-1 treated mice only show an increase in the CD226+ NK cells. As CD226 signalling has been shown to be critical for anti-tumor activity, the increase in CD226+ cells in both the CD8 and the NK cell population in the Antibody A treatment group may indicate the potential for enhanced cytotoxicity, which could contribute to the anti-tumor activity of Antibody A observed in the study.

TABLE 11
% CD226 positive CD8 T Cells

| | | | | Anti-human | |
|---|---|---|---|---|---|
| | | Anti- | | PD-1- | |
| | | human | Anti- | hIgG4-PAA + | |
| | | PD-1- | human | Anti-human | |
| | | hIgG4- | TIGIT- | TIGIT- | |
| | IgG | PAA | hIgG1-EN | hIgG1-EN | Antibody A |
| N 1 | 21.4 | 37.5 | 32.1 | 40 | 56.9 |
| N 2 | 23.9 | 82.2 | 53.6 | 20.9 | 64.3 |
| N 3 | 30.2 | 50 | 47 | 17.7 | 60 |
| N 4 | 33 | 60.3 | 22.1 | 57.1 | 50 |
| N 5 | 26.6 | 31.5 | 15 | 36 | 28.6 |
| N 6 | 59.3 | 40.6 | 48.5 | 75 | 50 |
| N 7 | 45.4 | 32.1 | 35.1 | 57.5 | 60 |
| N 8 | 45.1 | 56.2 | 19.9 | 23 | |
| mean | 35.6 | 48.8 | 34.2 | 40.9 | 52.8 |
| SE | 4.6 | 6.1 | 5.1 | 7.3 | 4.5 |
| p value | NA | p = .107 | p = .837 | p= .551 | p = .020 |
| vs IgG | | | | | |

TABLE 12
% CD226 positive NK Cells

| | | Anti- | | Anti-PD-1- | |
|---|---|---|---|---|---|
| | | human | Anti- | hIgG4- | |
| | | PD-1- | human | PAA + Anti- | |
| | | hIgG4- | TIGIT- | human TIGIT- | |
| | IgG | PAA | hIgG1-EN | hIgG1-EN | Antibody A |
| N 1 | 40 | 53.1 | 47.2 | 60.9 | 49.1 |
| N 2 | 49.9 | 61 | 60.3 | 22.1 | 62.7 |
| N 3 | 45.2 | 59.8 | 50.3 | 54.2 | 66.7 |
| N 4 | 57 | 49.7 | 41.5 | 60 | 72 |
| N 5 | 57.1 | 57.7 | 33.9 | 61.8 | 72.2 |
| N 6 | 49.5 | 49.6 | 58.1 | 71.6 | 88.4 |
| N 7 | 46.1 | 54.3 | 56.3 | 68.4 | 89.4 |
| N 8 | 49.8 | 63.6 | 29.3 | 56.6 | |
| mean | 49.3 | 56.1 | 47.1 | 57.0 | 71.5 |
| SE | 2.0 | 1.9 | 4.0 | 5.4 | 5.4 |
| p value | NA | p = .028 | p = .633 | p = .206 | p = .0013 |
| vs IgG | | | | | |

6 days post final dose, serum levels of the anti-human PD-1-hIgG4-PAA, anti-human TIGIT-hIgG1-EN and Antibody A are analyzed via ELISA. Recombinant human PD-1-his (R&D Systems, Cat: 8986-PD) and recombinant human TIGIT-his (R&D Systems, Cat: 9525-TG) are used for the PD-1 and TIGIT capture ELISAs respectively. Mouse anti-human IgG Fc HRP (Southern Biotech/9040-05) is used for detection.

In experiments performed essentially as described above, the results in Table 13 demonstrate that Antibody A serum levels as measured by both the human PD-1 and human TIGIT antigen capture ELISAs are comparable, thus suggesting in vivo stability of the antibody.

TABLE 13

| | Antibody Serum |
|---|---|
| Treatment Groups (Analyte) | Levels (ng/mL) |
| Anti-human PD-1-hIgG4-PAA (PD-1) | 137,242 |
| Anti-human PD-1-hIgG4-PAA (PD-1) | 214,785 |
| Anti-human PD-1-hIgG4-PAA (PD-1) | 260,079 |
| Anti-human PD-1-hIgG4-PAA (PD-1) | 271,484 |
| Anti-human PD-1-hIgG4-PAA (PD-1) | 179,951 |
| Anti-human PD-1-hIgG4-PAA (PD-1) | 144,798 |
| Anti-human PD-1-hIgG4-PAA (PD-1) | 148762 |
| Anti-human PD-1-hIgG4-PAA (PD-1) | 207223 |
| Average | 195541 |
| SD | 51885 |
| Anti-human TIGIT-hIgG1-EN (TIGIT) | 69087 |
| Anti-human TIGIT-hIgG1-EN (TIGIT) | 90291 |
| Anti-human TIGIT-hIgG1-EN (TIGIT) | 94828 |
| Anti-human TIGIT-hIgG1-EN (TIGIT) | 97295 |
| Anti-human TIGIT-hIgG1-EN (TIGIT) | 91847 |
| Anti-human TIGIT-hIgG1-EN (TIGIT) | 55174 |
| Anti-human TIGIT-hIgG1-EN (TIGIT) | 52438 |
| Anti-human TIGIT-hIgG1-EN (TIGIT) | 87853 |
| Average | 79852 |
| SD | 18212 |
| Antibody A (PD-1) | 96794 |
| Antibody A (PD-1) | 135103 |
| Antibody A (PD-1) | 152474 |
| Antibody A (PD-1) | 144320 |
| Antibody A (PD-1) | 107847 |
| Antibody A (PD-1) | 116441 |
| Antibody A (PD-1) | 160017 |
| Antibody A (PD-1) | 169076 |
| Average | 135259 |
| SD | 25967 |
| Antibody A (TIGIT) | 92771 |
| Antibody A (TIGIT) | 99719 |
| Antibody A (TIGIT) | 101530 |
| Antibody A (TIGIT) | 87067 |
| Antibody A (TIGIT) | 66332 |
| Antibody A (TIGIT) | 85640 |
| Antibody A (TIGIT) | 89285 |
| Antibody A (TIGIT) | 139837 |
| Average | 95272 |
| SD | 20992 |

Amino Acid and Nucleotide Sequences
(TIGIT HCDR1 amino acid sequence)
SEQ ID NO: 1
AASGFDFSSYGVP
(TIGIT HCDR2 amino acid sequence)
SEQ ID NO: 2
YIDPIFGPTYYADEVKG
(TIGIT HCDR3 amino acid sequence)
SEQ ID NO: 3
ARDYSYGYAYALDI
(TIGIT LCDR1 amino acid sequence)
SEQ ID NO: 4
QASQRISPYLA
(TIGIT LCDR2 amino acid sequence)
SEQ ID NO: 5
SRASKLAS
(TIGIT LCDR3 amino acid sequence)
SEQ ID NO: 6
QSYYVHTSSGYA
(PD-1 HCDR1 amino acid sequence)
SEQ ID NO: 7
KASGGTFSSYAIS
(PD-1 HCDR2 amino acid sequence)
SEQ ID NO: 8
LIIPSFDTAGYAQKFQG
(PD-1 HCDR3 amino acid sequence)
SEQ ID NO: 9
ARAEHSSTGTFDY
(PD-1 LCDR1 amino acid sequence)
SEQ ID NO: 10
RASQGISSWLA
(PD-1 LCDR2 amino acid sequence)
SEQ ID NO: 11
SAASSLQS
(PD-1 LCDR3 amino acid sequence)
SEQ ID NO: 12
QQANHLPFT
(TIGIT HCVR amino acid sequence)
SEQ ID NO: 13
EVQLVESGGGLVQPGGSLRLSCAASGFDFSSYGVPWVRKAPGKGLEWVGY
IDPIFGPTYYADEVKGRFTISADDSKNSLYLQMNSLKTEDTAVYYCARDY
SYGYAYALDIWGQGTLVTVSS
(TIGIT LCVR amino acid sequence)
SEQ ID NO: 14
RIVMTQTPLSLSVTPGQPASISCQASQRISPYLAWYLDKPGQPPQLLISR
ASKLASGVPDRFSGSGSGTDFTLKISRVEAEDVGVYYCQSYYVHTSSGYA
FGGGTKVEIK
(TIGIT HCCR amino acid sequence)
SEQ ID NO: 15
ASTKGPSVFPLAPSSKSTSGGTAALGCLVADYFPEPVTVSWNSGALTSGV
HTFPAVLQSSGLYSLSSVVTVPSSSLGTQTYICNVNHKPSNTKVDERVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVYTLPPSRGDMTKNQVQLTC
LVKGFYPSDIAVEWESNGQPENNYKTTPPVLDSDGSFFLASKLTVDKSRW
QQGNVFSCSVMHEALHNHYTQKSLSLSPGK
(TIGIT LCCR amino acid sequence)
SEQ ID NO: 16
RTVAAPSVFIFPPSDKQLKSGTARVVCLLNNFYPREAKVQWKVDNALQSG
NSQESVTEQDSKDSTYSLSSTLTLSKADYEKHKVYACEVTHQGLSSPVTK
SFNRGEC
(PD-1 HCVR amino acid sequence)
SEQ ID NO: 17
QVQLVQSGAEVKKPGSSVKVSCKASGGTFSSYAISWVRYAPGQGLEWMGL
IIPSFDTAGYAQKFQGRVAITVDESTSTAYMELSSLRSEDTAVYYCARAE
HSSTGTFDYWGRGTLVTVSS
(PD-1 LCVR amino acid sequence)
SEQ ID NO: 18
DIQMTQSPSSVSASVGDRVTITCRASQGISSWLAWYQRKPGDAPKLLISA
ASSLQSGVPSRFSGSGSGTDFTLTISSLQPEDFATYYCQQANHLPFTFGG
GTKVEIK
(PD-1 HCCR amino acid sequence)
SEQ ID NO: 19
ASTKGPSVFPLAPSSKSTSGGTAALGCLVKDYFPEPVTVSWNSGALTSGV
ATGPAVLQSSGLYSLSSVVTVPSSSLGTQTYICNVNHKPSNTKVDKRVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVSTLPPSREEMTKNQVSLMC
LVYGFYPSDIAVEWESNGQPENNYKTTPPVLDSDGSFFLYSVLTVDKSRW
QQGNVFSCSVMHEALHNHYTQKSLSLSPGK
(PD-1 LCCR amino acid sequence)
SEQ ID NO: 20
GQPKAAPSVTLFPPSSEELQANKATLVCYISDFYPGAVTVAWKADSSPVK
AGVETTTPSKQSNNKYAAWSYLSLTPEQWKSHRSYSCQVTHEGSTVEKTV
APTEC
(TIGIT HC amino acid sequence)
SEQ ID NO: 21
EVQLVESGGGLVQPGGSLRLSCAASGFDFSSYGVPWVRKAPGKGLEWVGY
IDPIFGPTYYADEVKGRFTISADDSKNSLYLQMNSLKTEDTAVYYCARDY
SYGYAYALDIWGQGTLVTVSSASTKGPSVFPLAPSSKSTSGGTAALGCLV
ADYFPEPVTVSWNSGALTSGVHTFPAVLQSSGLYSLSSVVTVPSSSLGTQ
TYICNVNHKPSNTKVDERVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALAAPIEKTISKAKGQPREP
QVYTLPPSRGDMTKNQVQLTCLVKGFYPSDIAVEWESNGQPENNYKTTPP
VLDSDGSFFLASKLTVDKSRWQQGNVFSCSVMHEALHNHYTQKSLSLSPG
K
(TIGIT LC amino acid)
SEQ ID NO: 22
RIVMTQTPLSLSVTPGQPASISCQASQRISPYLAWYLDKPGQPPQLLISR
ASKLASGVPDRFSGSGSGTDFTLKISRVEAEDVGVYYCQSYYVHTSSGYA
FGGGTKVEIKRTVAAPSVFIFPPSDKQLKSGTARVVCLLNNFYPREAKVQ
WKVDNALQSGNSQESVTEQDSKDSTYSLSSTLTLSKADYEKHKVYACEVT
HQGLSSPVTKSFNRGEC
(PD-1 HC amino acid sequence)
SEQ ID NO: 23
QVQLVQSGAEVKKPGSSVKVSCKASGGTFSSYAISWVRYAPGQGLEWMGL
IIPSFDTAGYAQKFQGRVAITVDESTSTAYMELSSLRSEDTAVYYCARAE
HSSTGTFDYWGRGTLVTVSSASTKGPSVFPLAPSSKSTSGGTAALGCLVK
DYFPEPVTVSWNSGALTSGVATGPAVLQSSGLYSLSSVVTVPSSSLGTQT
YICNVNHKPSNTKVDKRVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALAAPIEKTISKAKGQPREPQ
VSTLPPSREEMTKNQVSLMCLVYGFYPSDIAVEWESNGQPENNYKTTPPV
LDSDGSFFLYSVLTVDKSRWQQGNVFSCSVMHEALHNHYTQKSLSLSPGK
(PD-1 LC amino acid sequence)
SEQ ID NO: 24
DIQMTQSPSSVSASVGDRVTITCRASQGISSWLAWYQRKPGDAPKLLISA
ASSLQSGVPSRFSGSGSGTDFTLTISSLQPEDFATYYCQQANHLPFTFGG
GTKVEIKGQPKAAPSVTLFPPSSEELQANKATLVCYISDFYPGAVTVAWK
ADSSPVKAGVETTTPSKQSNNKYAAWSYLSLTPEQWKSHRSYSCQVTHEG
STVEKTVAPTEC
(TIGIT HC DNA sequence)
SEQ ID NO: 25
ATGGAGACGGACACTCTGCTCCTGTGGGTGCTCCTGCTTTGGGTACCGGG
TTCAACGGGAGAGGTGCAGCTGGTGGAGTCTGGGGGAGGCTTGGTCCAGC
CTGGAGGGTCCCTGAGACTCTCCTGTGCTGCTTCTGGATTCGACTTCAGT
AGTTATGGAGTGCCCTGGGTCCGCAAGGCTCCAGGGAAGGGGCTGGAGTG
GGTTGGCTACATTGATCCTATTTTTGGTCCCACATACTACGCAGACGAGG
TGAAGGGCAGATTCACCATCTCAGCTGATGATTCAAAGAACTCACTGTAT
CTGCAAATGAACAGCCTGAAAACCGAGGACACGGCCGTGTATTACTGTGC
GAGAGACTATAGTTATGGTTATGCTTATGCTCTCGACATCTGGGGCCAGG
GAACCCTGGTCACCGTCTCCTCAGCTAGCACCAAGGGCCCATCGGTCTTC
CCCCTGGCACCCTCCTCCAAGAGCACCTCTGGGGGCACAGCGGCCCTGGG
CTGCCTGGTCGCCGACTACTTCCCCGAACCGGTGACGGTGTCGTGGAACT
CAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTT
GGGCACCCAGACCTACATCTGCAACGTGAATCACAAGCCCAGCAACACCA
AGGTGGACGAGAGAGTTGAGCCCAAATCTTGTGACAAAACTCACACATGC
CCACCGTGCCCAGCACCTGAAGCCGCAGGGGGACCGTCAGTCTTCCTCTT
CCCCCCAAAACCCAAGGACACCCTCATGATCTCCCGGACCCCTGAGGTCA
CATGCGTGGTGGTGGACGTGAGCCACGAAGACCCTGAGGTCAAGTTCAAC
TGGTATGTGGACGGCGTGGAGGTGCATAATGCCAAGACAAAGCCGCGGGA
GGAGCAGTACAACAGCACGTACCGTGTGGTCAGCGTCCTCACCGTCCTGC
ACCAAGACTGGCTGAATGGCAAGGAGTACAAGTGCAAGGTCTCCAACAAA
GCCCTCGCCGCCCCCATCGAGAAAACCATCTCCAAAGCCAAAGGGCAGCC
CCGAGAACCACAGGTGTACACCCTGCCCCCATCCCGGGGGGACATGACCA
AGAACCAAGTCCAGCTGACCTGCCTGGTCAAAGGCTTCTATCCCAGCGAC
ATCGCCGTGGAGTGGGAGAGCAATGGGCAGCCGGAGAACAACTACAAGAC
CACGCCTCCCGTGCTGGACTCCGACGGCTCCTTCTTCCTCGCTTCCAAGC
TCACCGTGGACAAGAGCAGGTGGCAGCAGGGGAACGTCTTCTCATGCTCC
GTGATGCATGAGGCTCTGCACAACCACTACACGCAGAAGAGCCTCTCCCT
GTCTCCGGGCAAA
(TIGIT LC DNA sequence)
SEQ ID NO: 26
ATGGAAACTGACACCCTGCTGCTCTGGGTACTGCTCCTTTGGGTTCCTGG
GAGCACAGGCCGGATTGTGATGACCCAGACTCCACTCTCTCTGTCCGTCA
CCCCTGGACAGCCGGCCTCCATCTCCTGCCAGGCCAGTCAGAGAATTAGT
CCCTACTTAGCCTGGTACCTGGACAAGCCAGGCCAGCCTCCACAGCTCCT
GATCTCCCGGGCATCCAAACTGGCATCTGGAGTGCCAGATAGGTTCAGTG
GCAGCGGGTCAGGGACAGATTTCACACTGAAAATCAGCCGGGTGGAGGCT
GAGGATGTTGGGGTTTATTACTGCCAAAGTTATTATGTTCACACTAGTAG
TGGTTATGCTTTCGGCGGAGGGACCAAGGTGGAGATCAAACGGACCGTGG
CTGCACCATCTGTCTTCATCTTCCCGCCATCTGATAAGCAGTTGAAATCT
GGAACTGCCAGAGTTGTGTGCCTGCTGAATAACTTCTATCCCAGAGAGGC
CAAAGTACAGTGGAAGGTGGATAACGCCCTCCAATCGGGTAACTCCCAGG
AGAGTGTCACAGAGCAGGACAGCAAGGACAGCACCTACAGCCTCAGCAGC
ACCCTGACGCTGAGCAAAGCAGACTACGAGAAACACAAAGTCTACGCCTG
CGAAGTCACTCATCAGGGCCTGAGCTCGCCCGTCACAAAGAGCTTCAACA
GGGGAGAGTGC
(PD-1 HC DNA sequence)
SEQ ID NO: 27
ATGGAAACCGATACGCTCCTGCTGTGGGTTCTCCTCTTGTGGGTCCCCGG
CTCTACCGGGCAGGTCCAGCTCGTGCAGAGTGGCGCCGAGGTCAAAAAAC
CCGGTTCAAGCGTGAAGGTGTCTTGTAAAGCATCTGGAGGAACCTTTAGT
TCCTACGCCATTAGTTGGGTGAGGTACGCTCCCGGCCAGGGCTTGGAATG
GATGGGTTTGATTATTCCCAGCTTTGATACAGCTGGATACGCGCAGAAGT
TCCAGGGACGCGTGGCCATCACCGTGGATGAAAGCACTTCAACTGCCTAC
ATGGAACTGTCATCCTTGAGAAGCGAGGATACTGCTGTTTACTACTGCGC
TAGGGCAGAGCACTCCTCCACCGGGACCTTCGACTATTGGGGTCGAGGTA
CTCTCGTGACCGTGAGCAGCGCTAGCACCAAGGGCCCATCGGTCTTCCCC
CTGGCACCCTCCTCCAAGAGCACCTCTGGGGGCACAGCGGCCCTGGGCTG
CCTGGTCAAGGACTACTTCCCCGAACCGGTGACGGTGTCGTGGAACTCAG
GCGCCCTGACCAGCGGCGTGGCCACCGGCCCGGCTGTCCTACAGTCCTCA
GGACTCTACTCCCTCAGCAGCGTGGTGACCGTGCCCTCCAGCAGCTTGGG
CACCCAGACCTACATCTGCAACGTGAATCACAAGCCCAGCAACACCAAGG
TGGACAAGAGAGTTGAGCCCAAATCTTGTGACAAAACTCACACATGCCCA
CCGTGCCCAGCACCTGAAGCCGCAGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TATGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AAGACTGGCTGAATGGCAAGGAGTACAAGTGCAAGGTCTCCAACAAAGCC
CTCGCCGCCCCCATCGAGAAAACCATCTCCAAAGCCAAAGGGCAGCCCCG
AGAACCACAGGTGTCCACCCTGCCCCCATCCCGGGAGGAGATGACCAAGA
ACCAAGTCAGCCTGATGTGCCTGGTCTATGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATTCCGTGCTCA
CCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTC
TCCGGGCAAA
(PD-1 LC DNA sequence)
SEQ ID NO: 28
ATGGAGACAGACACACTCCTGCTATGGGTACTGCTGCTCTGGGTTCCAGG
ATCCACTGGTGACATCCAGATGACACAGTCACCTTCAAGCGTCTCCGCCT
CCGTGGGAGACAGGGTTACTATTACATGTAGGGCCAGCCAGGGGATCTCT
TCATGGCTGGCGTGGTACCAACGGAAGCCAGGCGACGCCCCCAAGCTCCT
TATCTCCGCTGCCTCCTCTCTGCAGTCCGGAGTTCCCTCCCGCTTCAGCG
GTAGCGGGTCAGGCACTGACTTCACCCTTACAATCTCTTCTCTGCAACCT
GAGGACTTCGCCACATATTATTGCCAGCAGGCAAACCATTTGCCATTTAC
TTTTGGCGGAGGTACTAAGGTTGAGATTAAAGGCCAGCCTAAAGCTGCCC
CTAGCGTTACCCTTTTCCCACCGAGCTCCGAGGAGCTGCAGGCCAATAAA
GCAACCTTGGTCTGCTACATATCAGATTTTTACCCTGGCGCCGTGACCGT
AGCATGGAAAGCTGATTCATCCCCTGTGAAGGCCGGTGTTGAAACTACAA
CCCCTTCCAAACAATCTAACAATAAATACGCGGCATGGTCCTACCTGTCC
TTGACACCCGAGCAGTGGAAATCTCACAGATCTTACAGCTGCCAGGTCAC
CCACGAGGGGAGCACTGTGGAGAAGACCGTCGCGCCCACTGAGTGC
(Human PD-1 amino acid sequence)
SEQ ID NO: 29
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTA
GARRTGQPLKEDPSAVPVFSVDYGELDFQWREKTPEPPVPCVPEQTEYAT
IVFPSGMGTSSPARRGSADGPRSAQPLRPEDGHCSWPL
(Human PD-1 ECD-His amino acid sequence)
SEQ ID NO: 30
LDSPDRPWNPPTFSPALLVVTEGDNATFTCSFSNTSESFVLNWYRMSPSN
QTDKLAAEPEDRSQPGQDCRFRVTQLPNGRDFHMSVVRARRNDSGTYLCG
AISLAPKAQIKESLRAELRVTERRAEVPTAHPSPSPRPAGQFQHHHHHH
(Human TIGIT amino acid sequence)
SEQ ID NO: 31
MRWCLLLIWAQGLRQAPLASGMMTGTIETTGNISAEKGGSIILQCHLSST
TAQVTQVNWEQQDQLLAICNADLGWHISPSFKDRVAPGPGLGLTLQSLTV
NDTGEYFCIYHTYPDGTYTGRIFLEVLESSVAEHGARFQIPLLGAMAATL
VVICTAVIVVVALTRKKKALRIHSVEGDLRRKSAGQEEWSPSAPSPPGSC
VQAEAAPAGLCGEQRGEDCAELHDYFNVLSYRSLGNCSFFTETG
(Human TIGIT ECD-His amino acid sequence)
SEQ ID NO: 32
HEIHHEIRGGGGSMMTGTIETTGNISAEKGGSIILQCHLSSTTAQVTQVN
WEQQDQLLAICNADLGWHISPSFKDRVAPGPGLGLTLQSLTVNDTGEYFC
IYHTYPDGTYTGRIFLEVLESSVAEHGARFQIPGGGGSHHHHHH

1. A polypeptide molecule that binds to human TIGIT (SEQ ID NO:31), comprising the amino acid sequences of SEQ ID NOS:1-6.

2. The polypeptide molecule of claim 1, further comprising the amino acid sequences of SEQ ID NOS: 7-12, wherein the polypeptide molecule also binds to human PD-1 (SEQ ID NO: 29).

3. The polypeptide molecule of claim 1, wherein the polypeptide molecule is an scFv molecule.

4. The polypeptide molecule of claim 3, wherein the scFv molecule is a polyspecific scFv molecule.

5. The polypeptide molecule of claim 4, wherein the polyspecific scFv molecule is a bispecific scFv molecule.

6. The polypeptide molecule of claim 1, wherein the polypeptide molecule is an antibody, or a TIGIT-binding fragment thereof.

7. The polypeptide molecule of claim 6, wherein the polypeptide molecule is an antibody.

8. The polypeptide molecule of claim 7, wherein the antibody is a mono-specific antibody.

9. The polypeptide molecule of claim 7, wherein the polypeptide molecule is a polyspecific antibody.

10. The polypeptide molecule of claim 9, wherein the polypeptide molecule is a bispecific antibody.

11. The polypeptide molecule of claim 6, wherein the antibody or a TIGIT-binding fragment thereof comprises a heavy chain variable region having the amino acid sequence of SEQ ID NO: 13 and a light chain variable region having the amino acid sequence of SEQ ID NO: 14.

12. The polypeptide molecule of claim 7, wherein the antibody comprises a heavy chain having the amino acid sequence of SEQ ID NO: 21 and a light chain having the amino acid sequence of SEQ ID NO: 22.

13. The polypeptide molecule of claim 6, wherein the antibody or TIGIT-binding fragment thereof also binds to human PD-1 (SEQ ID NO: 29) and further comprises the amino acid sequences of SEQ ID NOS: 7-12.

14. The polypeptide molecule of claim 13, wherein the polypeptide molecule is an antibody, or a human TIGIT and human PD-1 binding fragment thereof, comprising:
a) a first heavy chain variable region having the amino acid sequence of SEQ ID NO: 13;
b) a first light chain variable region having the amino acid sequence of SEQ ID NO: 14;
c) a second heavy chain variable region having the amino acid sequence of SEQ ID NO: 17; and
d) a second light chain variable region having the amino acid sequence of SEQ ID NO: 18.

15. The polypeptide molecule of claim 14, wherein the polypeptide molecule is an antibody comprising:
a) a first heavy chain having the amino acid sequence of SEQ ID NO:21;
b) a first light chain having the amino acid sequence of SEQ ID NO:22;
c) a second heavy chain having the amino acid sequence of SEQ ID NO:23; and
d) a second light chain having the amino acid sequence of SEQ ID NO:24.

16. (canceled)

17. A DNA molecule comprising a polynucleotide encoding one or more of the amino acid sequences of SEQ ID NO:21, SEQ ID NO: 22, SEQ ID NO: 23 and SEQ ID NO: 24.

18. The DNA molecule of claim 17, wherein the polynucleotide further comprises one or more of the DNA sequences of SEQ ID NO:25, SEQ ID NO: 26, SEQ ID NO: 27 and SEQ ID NO: 28.

19. A mammalian cell comprising the DNA molecule of claim 17.

20. A process for producing an antibody, comprising cultivating the mammalian cell of claim 18, and recovering the polypeptide molecule.

21. The polypeptide molecule produced by the method of claim 20.

22. A pharmaceutical composition comprising the polypeptide molecule of claim 1, and an acceptable carrier, diluent, or excipient.

23. A method of treating a solid tumor cancer comprising administering to a human patient in need thereof, an effective amount of the polypeptide molecule of claim 1.

24. The method of claim 23, wherein the solid tumor cancer is lung cancer, breast cancer, head and neck cancer, melanoma, liver cancer, colorectal cancer, pancreatic cancer, gastric cancer, kidney cancer, prostate cancer, ovarian cancer, endometrial cancer, or hepatocellular carcinoma.

25.-27. (canceled)

28. The method of claim 1, wherein the polypeptide molecule is administered in simultaneous, separate, or sequential combination with ionizing radiation.

29. The method of claim 1, wherein the polypeptide molecule is administered in simultaneous, separate, or sequential combination with one or more chemotherapeutic agents.

30.-44. (canceled)

45. A mammalian cell comprising the DNA molecule of claim 18.
